(19) United States
(12) Patent Application Publication
(10) Pub. No.: US 2016/0089346 A1
(43) Pub. Date: Mar. 31, 2016

(54) METHOD OF TREATMENT FOR BODY CONTOURING

(71) Applicant: Neothetics, Inc., San Diego, CA (US)

(72) Inventor: Kenneth Walter Locke, San Diego, CA (US)

(21) Appl. No.: 14/866,471

(22) Filed: Sep. 25, 2015

Related U.S. Application Data
(60) Provisional application No. 62/055,438, filed on Sep. 25, 2014.
(60) Provisional application No. 62/055,440, filed on Sep. 25, 2014.

(51) Int. Cl. A61K 31/137; A61B 5/00; A61B 5/055; A61B 5/107

(52) U.S. Cl. CPC A61K 31/137; A61B 5/1077; A61B 5/4839; A61B 5/4848; A61B 5/4872; A61B 5/1079; A61B 5/1075; A61B 5/1072; A61B 5/055; A61B 5/0053; A61B 2019/5276

(57) ABSTRACT

Provided herein are methods involving body contouring applications including treatments to reduce subcutaneous fat. The methods include the determination of patient suitability for body contouring treatment by evaluating one or more patient assessments. In various implementations, a patient determined to be suitable for body contouring treatment is administered a composition comprising a beta-2 adrenergic receptor agonist to the region of the body comprising subcutaneous fat, such as the central abdominal or submental region.

### CROSS REFERENCE

This application claims the benefit of U.S. Provisional Application No. 62/055,438 filed Sep. 25, 2014 and U.S. Provisional Application No. 62/055,440 filed Sep. 25, 2014, which are hereby incorporated by reference in their entirety.

### BACKGROUND OF THE INVENTION

A reduction of whole body fat stores has traditionally been achieved by diet and/or exercise. Localized fat reduction is typically and has traditionally been achieved by surgically removing fat by methods such as lipoplasty or liposuction, or by non-surgical methods such as energy-based medical devices.

### SUMMARY OF THE INVENTION

Fat reduction and body contouring medical procedures are highly sought after methods that serve as alternatives or supplements to diet and exercise for achieving a desired physique. Current treatment options to address this patient demand are limited to surgical options and non-surgical options designed to damage or kill fat cells, which in many cases can cause adverse consequences for the patient. For instance, while surgical options effectively reduce fat, they require significant physician skill and resources, may involve pain, downtime, and expense for the patient, and carry the risks associated with any surgical procedure. Existing non-surgical options can often be painful, produce limited or inconsistent results, cause nerve damage, burns and scarring, and require multiple treatments, ongoing maintenance treatments, or special patient diet or exercise programs. The present disclosure describes an alternative to the current treatment methods by locally administering a pharmaceutical composition to a desired region of the body, resulting in fat reduction in the desired region with minimal risk and limited to no recovery time.

In one aspect of the disclosure, provided are body contouring methods that reduce bulging due to subcutaneous fat and/or a visual appearance of fat due to excess subcutaneous tissue in a desired treatment region of a patient's body. In some embodiments, body contouring methods comprise determining patient suitability for body contouring treatment by one or more assessments performed on the patient by the patient, a clinician, or both the patient and clinician. In some embodiments, body contouring methods comprise administering a composition comprising a beta-2 adrenergic receptor agonist to the desired treatment region of the patient after the patient has been determined to be suitable for body contouring treatment by the one or more assessments. The therapeutic effectiveness of administering said composition is determined by evaluating the treatment region prior to and after administration, for example, by visual assessment and/or a direct or indirect measurement of bulging due to excess subcutaneous fat. Treatment regions include regions of the body having visible and/or protruding amounts of subcutaneous fat, including, without limitation, head and neck (including face), flank, abdomen, back, chest, arm, leg and buttock. The head and neck region includes, without limitation, orbit, periorbit, neck, chin and submental region. In some cases, the treatment region is a central abdominal region, which is often diagnosed as having central abdominal bulging by the one or more assessments. In some cases, the treatment region is a submental region.

In one aspect of the disclosure, provided are methods for determining patient suitability for body contouring treatment by performing one or more assessments on the patient. In another aspect, provided are body contouring treatment methods comprising administering to a patient a composition comprising a long-acting beta-2 adrenergic receptor agonist to a treatment region of the patient's body, provided that the patient has been identified as suitable for body contouring treatment as determined by one or more assessments. In some embodiments, a patient suitability assessment comprises determining if a patient is non-obese. In some embodiments, a patient suitability assessment comprises determining if the patient has subcutaneous fat in the treatment region of the body. In some embodiments, a patient suitability assessment comprises measuring, either directly or indirectly, the amount of subcutaneous fat or bulging due to subcutaneous fat in the desired treatment region of the patient. Exemplary measurement techniques for measuring subcutaneous fat or bulging due to subcutaneous fat include, without limitation, the use of a caliper, tape measure, magnetic resonance imaging and two-dimensional ultrasound. In some cases, the tape measure is guided by a laser. In some embodiments, a patient suitability assessment comprises measuring patient body mass index (BMI). In some embodiments, a patient suitability assessment comprises visual grading of the desired treatment region of the patient. Visual grading is performed by the patient, a clinician, or both the patient and clinician, and comprises grading the amount of visual bulging and/or rating the contour and/or appearance of fat in the treatment region using descriptive terms and/or numerals. In many cases, the appearance of fat is a visible protrusion of fat and/or skin from the body region. In some instances, the descriptive terms and/or numerals are selected by comparing the patient's body region to a set of pictures of corresponding reference regions, wherein each reference picture is assigned a descriptive term and/or number. In some embodiments, a patient suitability assessment comprises palpation of the region by a clinician. In some embodiments, a patient suitability assessment comprises three-dimensional digital photographic imaging. In some embodiments, a patient suitability assessment comprises performing magnetic resonance imaging. In some embodiments, a patient suitability assessment comprises two-dimensional ultrasound. In some embodiments, a patient suitability assessment comprises measurement with a tape measure. In some cases, the tape measurement is guided by a laser. In some embodiments, a patient suitability assessment comprises subjective evaluation by the patient, clinician, or both the patient and clinician.

In another aspect of the disclosure, provided are methods for determining the effect of a body contouring treatment administered to a region of the body of a patient, comprising performing one or more assessments to evaluate, directly or indirectly, a reduction of subcutaneous fat and/or a reduction of the appearance of fat due to subcutaneous fat in said region, provided that at least one assessment is conducted after administration to the region a composition comprising an effective amount of a long-acting beta-2 adrenergic receptor agonist. In some embodiments, at least one assessment is conducted before composition administration. In some embodiments, the region of the body comprises subcutaneous fat prior to administration. In various embodiments, the region of the body is a submental region. In some embodiments, the region of the body is a central abdominal region. In some cases, the treatment is administered to the patient if the patient is non-obese. In some cases, a non-obese patient is one that does not have a BMI greater than about 30 kg/m². In some embodiments, a patient suitable for body contouring treatment does not have a BMI less than about 18 kg/m².

Treatment region assessment is performed prior to, during the course of, and/or after treatment of the region. In some cases, the assessment is a patient suitability assessment to determine if the patient is suitable for body contouring treatment methods. In some cases, the assessment is performed to monitor the effectiveness of a body contouring treatment method. Body contouring treatment includes, without limitation, one or more sessions wherein a composition comprising a beta-2 adrenergic receptor agonist is administered to the region of the body. In cases wherein the assessment is performed to monitor the effectiveness of the treatment, the assessment may be performed from about 1 day to about 52 weeks after the final administration of the composition in a treatment period.

A patient suitable for body contouring treatment, in various embodiments, has a visible appearance of fat in the treatment region due to excess subcutaneous fat. A region suitable for body contouring treatment, in some instances, comprises less than about 8, 7, 6, 5, 4, 3 or 2 cm of subcutaneous fat. In some embodiments, the subcutaneous fat of the body region is superficial adiposity. In some embodiments, the fat is distinctly visible and palpable. In some embodiments, the patient does not have musculo skeletal abnormalities responsible for the appearance of bulging in the region.

In some embodiments, a patient suitable for body contouring treatment has bulging in the central abdominal region due to excess subcutaneous fat. In some cases, the central abdominal bulging is not caused by rectus diastasis or a hernia. In some embodiments, determining subcutaneous fat as central abdominal bulging comprises visualizing periumbilical soft tissue bulging in the patient, provided that the visualization is performed when the patient is assuming proper posture, the patient is not contracting their abdomen, and the patient has fully and naturally exhaled. In some embodiments, determining subcutaneous fat as central abdominal bulging comprises palpating the abdomen of the patient; provided that the patient is assuming proper posture, the patient is not contracting their abdomen, and the patient has fully and naturally exhaled. In some cases, a patient suitable for body contouring treatment has periumbilical bulging in the abdomen region due to an accumulation of excess subcutaneous fat. In some instances, the patient has periumbilical bulging that is distinctly visible and palpable. In some embodiments, the patient has periumbilical bulging covering an area less than about 600 cm² due to an accumulation of excess subcutaneous fat around their umbilicus.

In some embodiments, a patient suitable for body contouring treatment has submental fat. In many cases, the submental fat is due to excess subcutaneous fat. In some instances, the patient has submental fat that is distinctly visible and palpable. In some embodiments, the patient has submental fat due to an accumulation of excess subcutaneous fat covering an area less than about 400 cm². In some embodiments, the determination of submental fat as subcutaneous fat comprises visualizing submental soft tissue in the patient, provided that the visualization is performed when the patient is assuming proper head and neck posture. In some embodiments, the determination of submental fat as subcutaneous fat comprises palpating the submental region of the patient, provided that patient is assuming proper head and neck posture.

In some embodiments, an assessment of the treatment region comprises a patient self-assessment of the contour or appearance of the treatment region. In some embodiments, an assessment of the treatment region comprises a patient self-assessment of bulging in the treatment region. A patient self-assessment, in some instances, comprises evaluating the region using a descriptive phrase. In some embodiments, the descriptive phrase is converted to a number to generate a patient self-assessment score. In some embodiments, there are 3, 4, 5, 6, 7, 8, 9, 10 or more descriptive phrases. In some embodiments, there are five descriptive phrases. In some embodiments, the descriptive phrases are each converted to a different number ranging from a low score to a high score. As an example, the five descriptive phrases are converted to numbers 0 to 4. In some embodiments, a low patient self-assessment score, e.g., 0 on a scale of 0 to 4, indicates no visible fat in the region, a low level of visible fat in the region, and/or a low level of bulging in the region and a high score, e.g., 4 on a scale of 0 to 4, indicates a high level of visible fat and/or a high level of bulging in the region. A patient suitable for treatment, in various embodiments, has a patient self-assessment score greater than the lowest score, e.g., from 1 to 4 on a scale of 0 to 4. In some embodiments, following treatment, a patient self-assessment score has decreased by at least one point grade. For example, following treatment, a patient achieves the lowest score or second lowest score, e.g., 0 or 1 on a scale of 0 to 4, as determined by the patient self-assessment score.

In some embodiments, an assessment comprises a clinician assessment of the contour or appearance of the treatment region. In some embodiments, an assessment of the treatment region comprises a clinician assessment of bulging in the treatment region. In some embodiments, the clinician assessment comprises a match-to-sample task, wherein the clinician rates the appearance or bulging in the treatment region on a photonumeric scale. In some embodiments, the region is a submental region comprising subcutaneous fat. In some embodiments, the region is a central abdominal region. In some embodiments, the photonumeric scale comprises 3, 4, 5, 6, 7, 8, 9, 10 or more photographic images. In some cases, each of the photographic images corresponds to a different numerical point. As a non-limiting example, the photonumeric scale has six photographic images corresponding to six numerical points. In some embodiments, the photonumeric scale is a gender-specific scale. In some embodiments, the scale comprises representative pictures of increasing visible fat or bulging in a region or differential regional contours, with each picture corresponding to a different figure, e.g., number or letter. As a non-limiting example, the scale comprises 6 representative pictures of increasing visible fat or regional bulging rated on a scale from 0 to 5. In some embodiments, a lower number on the scale is indicative of a region having a lower amount of visible fat or bulging than a higher number on the scale. In some embodiments, a lower number on the scale is indicative of a region having a lower amount of subcutaneous fat or bulging than a higher number on the scale. In some embodiments, a patient suitable for treatment has a match-to-sample task score from 1 to 5, on a scale of 0 to 5. In some embodiments, following treatment, the match-to-sample task score decreases. For example, for a scale ranging from 0 to 5, the match-to-sample task score decreases by at least 1, 2 or 3 points following treatment.

In some embodiments, an assessment comprises a manual tape measure assessment. In some cases, the tape measure is guided by use of a laser. In some embodiments, the region of the body is a central abdominal region. In some embodiments, the laser-guided manual tape measure assessment is a measure of abdominal circumference. The abdominal circumference is measured, in various implementations, at one or more positions on the abdomen. In some embodiments, following treatment, the laser-guided manual tape measured value of bulging has decreased by at least about 1 cm as compared to the laser-guided manual tape measured value of bulging prior to treatment. In some embodiments, following treatment, the laser-guided manual tape measured value of bulging has decreased by at least about 1.5 cm as compared to the laser-guided manual tape measured value of bulging prior to treatment.

In some embodiments, an assessment comprises determining patient satisfaction with the treatment region. In some embodiments, a patient suitable for treatment has dissatisfaction with the contour of the treatment region. In some embodiments, the treatment region has subcutaneous fat or bulging. In some embodiments, the region is a submental region having excess subcutaneous fat. In some embodiments, the region has central abdominal bulging due to excess subcutaneous fat.

In some embodiments, an assessment comprises performing three-dimensional photographic imaging of the region of the patient. In some embodiments, the imaging is indicative of the region having bulging or excess subcutaneous fat.

In some embodiments, an assessment comprises directly or indirectly measuring subcutaneous fat in the region of the patient using a skin-fold caliper.

In some embodiments, an assessment comprises a subjective photographic assessment of the region of the patient having bulging or excess subcutaneous fat.

In some embodiments, an assessment comprises administering one or more questionnaires to the patient, a clinician, or both the patient and clinician.

In some embodiments, an assessment comprises performing magnetic resonance imaging of the body region of the patient having subcutaneous fat.

In some embodiments, an assessment comprises measuring the body region using a tape measure.

In some embodiments, an assessment comprises measuring the region using a laser-guided tape measure.

In some embodiments, an assessment comprises performing ultrasound imaging of the body region of the patient having subcutaneous fat.

In some embodiments, an assessment comprises administration of a body contour questionnaire completed by the patient. In some embodiments, at least one of the questions of the body contour questionnaire indicates patient satisfaction with the contour of the region.

Body contouring treatments described herein, in various instances, comprise the administration of a composition comprising a beta-2 adrenergic receptor agonist to a treatment region of the body in need of a reduction in the visual appearance of fat and/or bulging due to excess subcutaneous tissue in the region. In some cases, the beta-2 adrenergic receptor agonist comprises salmeterol, a salt or solvate of salmeterol, or a combination thereof. In some embodiments, the beta-2 adrenergic receptor agonist comprises salmeterol xinafoate. In some embodiments, the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof. In some embodiments, the beta-2 adrenergic receptor agonist is lipophilic. In some embodiments, the composition further comprises one or more compounds to prevent or reduce desensitization of beta adrenergic receptors. In some embodiments, the one or more compounds to prevent or reduce desensitization of beta-adrenergic receptors comprise a glucocorticosteroid. In some embodiments, the composition further comprises an antihistamine. In some embodiments, the antihistamine is ketotifen. In some embodiments, a compound configured to prevent or reduce desensitization of beta adrenergic receptors comprises fluticasone propionate. In some embodiments, a compound configured to prevent or reduce desensitization of beta adrenergic receptors comprises dexamethasone, prednisolone, methyl prednisolone, fluticasone, budesonide, or a salt, solvate, or combination thereof. In some embodiments, the composition comprises a sustained release formulation. In some embodiments, the composition is in a crystalline microparticle form. In some embodiments, the composition is in a lyophilized form. In some embodiments, the lyophilized form is reconstituted prior to administration.

In some embodiments, the composition comprising a beta-2 adrenergic receptor agonist is configured to reduce a regional fat deposit. In some embodiments, the composition is formulated for injection into a layer of subcutaneous fat. In some embodiments, the composition is formulated for transdermal administration to the region of the patient having subcutaneous fat. In some embodiments, the composition selectively partitions into adipose tissue relative to blood plasma after administration. In some embodiments, beta-2 adrenergic receptor agonist is configured to reduce subcutaneous fat. In some embodiments, the treatment is for cosmetic fat reduction. In some embodiments, subcutaneous fat reduction has a psychologically therapeutic benefit.

In some embodiments, the composition is administered to the region of the body having subcutaneous fat by subcutaneous injection. In some embodiments, the composition is administered to the region of the body having subcutaneous fat by transdermal administration. In some embodiments, the composition is administered to the region having subcutaneous fat in a dosage comprising from about 1 nanogram to about 50 micrograms beta 2-adrenergic receptor agonist. In some embodiments, each dosage is administered from about 1 to about 100 times to the region having subcutaneous fat during a session. In some embodiments, a total dosage administered during a session comprises from about 0.05 to about 500 micrograms beta-2 adrenergic receptor agonist. In some embodiments, a total dosage administered during a session comprises about 0.4 micrograms beta-2 adrenergic receptor agonist. In some embodiments, a total dosage administered during a session comprises about 0.4 micrograms salmeterol xinafoate. In some embodiments, a course of treatment comprises one session per week, for a period from about 2 to about 52 weeks. In some embodiments, a course of treatment comprises one session per week, for a period of about 8 weeks. In some embodiments, each dosage is administered from about 0.2 cm to about 10 cm apart to the region having subcutaneous fat. In some embodiments, each dosage is administered from about 1 cm to about 6 cm apart. In some embodiments, each dosage is administered about 4 cm apart. In some embodiments, the composition is administered to the abdomen. In some embodiments, the composition is administered between axial planes located at about +100 mm and about −150 mm, relative to the umbilicus. In some embodiments, the composition is administered between axial planes located at about +70 mm and about −90 mm, relative to the umbilicus. In some embodiments, the composition is administered between axial planes located at about +35 mm and about −70 mm, relative to the umbilicus. In some embodiments, the composition is administered to the submental region.

In some embodiments, the treatment region is defined by a temporary tattoo. In some embodiments, the temporary tattoo is configured to facilitate the administration of the composition to distinct treatment areas (or treatment sites) of the region. In some embodiments, the temporary tattoo is configured into a grid. In some embodiments, the temporary tattoo is water-based. In some embodiments, the temporary tattoo indicates from about 10 to about 100 positions on the region of the body for administration of the composition. In some embodiments, the composition is administered to one or more of the positions indicated by the temporary tattoo. In some embodiments, the region of the body is defined by a grid using a stencil. In this instance, the stencil is placed over the treatment region while the composition is administered at one or more distinct areas of the region, as defined by the stencil.

In some embodiments, the patient is not anesthetized before or during administration of the composition. In some embodiments, the administration of the composition does not result in contour irregularities. In some embodiments, the composition does not result in an inflammatory reaction.

In some embodiments, the administration of the composition results in attenuated regional fat accumulation. In some embodiments, the administration of the composition results in a decrease in regional adipose tissue.

In some embodiments, the administration of the composition improves the cosmetic appearance of the patient. In some embodiments, the administration of the composition is at least partially therapeutic. In some embodiments, the administration of the composition is at least partially cosmetic.

In some embodiments, the administration of the composition results in a decrease in subcutaneous fat in the region as compared to subcutaneous fat in the region prior to administration. In some embodiments, the administration of the composition results in a decrease in the appearance of fat or bulging in the region as compared to the appearance of fat in the region prior to administration. In some embodiments, the decrease in subcutaneous fat, as measured directly or indirectly, is at least about a 1% decrease in fat thickness or fat volume. Fat thickness or fat volume, in many instances, is measured using a caliper, ultrasound, magnetic resonance imaging, or a combination thereof.

In some embodiments, the administration of the composition to a central abdominal region results in a decrease in bulging due to subcutaneous fat in the region as compared to bulging in the region prior to administration. In some embodiments, the decrease is at least about a 1% decrease as measured using a manual tape measure. In some cases, the tape measurement is guided using a laser.

In another aspect, provided herein is a method of treatment for orbital proptosis or exophthalmos comprising administering to a patient a composition comprising a long-acting beta-2 adrenergic receptor agonist, provided that the patient has been identified as suitable for treatment by the performance of one or more assessments prior to treatment.

In a further aspect, provided herein is a method of determining the effect of a treatment in a patient for exophthalmos comprising performing one or more assessments to evaluate the reduction of exophthalmos in a patient after administration of a composition comprising an effective amount of a long-acting beta-2 adrenergic receptor agonist. The method further comprises performing one or more assessments before administration of the composition to the patient.

In some embodiments, the one or more assessments comprises three-dimensional digital photographic imaging. In some embodiments, the one or more assessments comprises a subjective photographic assessment. In some embodiments, the assessment comprises a measuring a physical characteristic. In some embodiments, the physical characteristic is exophthalmos. In some embodiments, the assessment comprises administering one or more questionnaires to the patient, a clinician, or both the patient and clinician. In some embodiments, the assessment comprises performing magnetic resonance imaging or computerized tomography scan of the orbit. In some embodiments, the one or more assessments comprises measuring proptosis using a Hertel exophthalmometer.

In some embodiments, the beta-2 adrenergic receptor agonist comprises salmeterol or a salt, solvate, or combination thereof. In some embodiments, the beta-2 adrenergic receptor agonist comprises salmeterol xinafoate. In some embodiments, the long-acting beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof. In some embodiments, the long-acting beta-2 adrenergic receptor agonist is salmeterol xinafoate and the composition further comprises fluticasone propionate. In some embodiments, the beta-2 adrenergic receptor agonist is lipophilic. In some embodiments, the composition further comprises one or more compounds to prevent or reduce desensitization of beta-2 adrenergic receptors. In some embodiments, the one or more compounds to prevent or reduce desensitization of beta adrenergic receptors comprises a glucocorticosteroid. In some embodiments, the one or more compounds to prevent or reduce desensitization of beta adrenergic receptors comprises an antihistamine. In some embodiments, the antihistamine is ketotifen. In some embodiments, the one or more compounds to prevent or reduce desensitization of beta adrenergic receptors comprises fluticasone propionate. In some embodiments, the one or more compounds to prevent or reduce desensitization of beta adrenergic receptors comprises dexamethasone, prednisolone, methyl prednisolone, fluticasone, budesonide, or a salt, solvate, or combination thereof. In some embodiments, the composition comprises a sustained release formulation. In some embodiments, the composition is in a crystalline microparticle form. In some embodiments, the composition is in lyophilized form. In some embodiments, the composition selectively partitions into adipose tissue relative to blood plasma after administration.

In some embodiments, the administration of the composition is at least partially therapeutic. In some embodiments, the administration of the composition is at least partially cosmetic. In some embodiments, the administration of the composition results in a decrease in exophthalmos as determined by one or more objective or subjective assessments.

In one aspect of the subject matter described herein, provided is a method for determining patient suitability for a body contouring treatment, the method comprising performing one or more assessments, provided that the one or more assessments comprises (a) determining if the patient is non-obese and (b) determining if the patient has subcutaneous fat in a treatment region of the patient's body; provided that the body contouring treatment comprises administration of a composition comprising a long-acting beta-2 adrenergic receptor agonist to the treatment region. In some embodiments, the treatment region comprises a submental region, central abdominal region, back, chest, arm, leg, buttock, face, flank, or combination thereof. In some embodiments, the treatment region is a central abdominal region comprising less than about 600 cm² subcutaneous fat around the umbilicus; a submental region comprising less than about 400 cm² subcutaneous fat; or a combination thereof. In some embodiments, the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof. In some embodiments, the beta-2 adrenergic receptor agonist comprises salmeterol xinafoate. In some embodiments, the composition further comprises one or more compounds to prevent or reduce desensitization of f3-adrenergic receptors, an antihistamine, or a combination thereof. In some embodiments, the one or more compounds to prevent or reduce desensitization of β-adrenergic receptors comprises a glucocorticosteroid.

In some embodiments, the one or more assessments comprises evaluating visible fat protrusion or bulging in the treatment region by selecting a descriptive phrase to describe the visible fat protrusion or bulging from an assessment scale comprising from about 3 to about 10 descriptive phrases that each indicate varying degrees of visible fat protrusion or bulging. In some embodiments, each descriptive phrase of the assessment scale is converted to a number on an ordinal scale to generate an assessment score; and provided that the lowest assessment score indicates no or a low level of visible fat protrusion or bulging and the highest assessment score indicates a high level of visible fat protrusion or bulging. In some embodiments, a patient suitable for treatment has an assessment score greater than the lowest assessment score.

In some embodiments, the one or more assessments comprises performing a match-to-sample task comprising rating visible fat protrusion or bulging in the treatment region of the patient on a photonumeric scale comprising from about 3 to about 10 representative pictures of increasing visible fat protrusion or bulging in corresponding reference treatment regions, each picture having a numeric value directly proportional to amount of visible fat protrusion or bulging. In some embodiments, a patient suitable for treatment has a match-to-task score greater than the lowest numeric value of the photonumeric scale.

In some embodiments, the one or more assessments comprises measuring the treatment region using a laser-guided manual tape measure; three-dimensional digital photographic imaging of the treatment region; measuring the treatment region using a skin-fold caliper; subjective photographic assessment of the treatment region; administering one or more questionnaires to the patient, a clinician, or both the patient and clinician; performing magnetic resonance imaging of the treatment region; performing ultrasound imaging of the treatment region; visual grading; palpating the treatment region; or a combination thereof.

In another aspect of the subject matter described herein, provided is a method of reducing subcutaneous adipose tissue, the method comprising administering to a treatment region of a patient a composition comprising a long-acting beta-2 adrenergic receptor agonist, provided that the patient has been identified as suitable for reducing subcutaneous adipose tissue or bulging due to subcutaneous adipose tissue in the treatment region as determined by one or more assessments, provided that the one or more assessments comprises (a) determination that the patient is non-obese and (b) determination of subcutaneous fat or bulging in the treatment region. In some embodiments, the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof. In some embodiments, the beta-2 adrenergic receptor agonist comprises salmeterol xinafoate. In some embodiments, the composition further comprises one or more compounds to prevent or reduce desensitization of a beta-adrenergic receptor, an antihistamine, or a combination thereof. In some embodiments, the one or more compounds to prevent or reduce desensitization of the beta-adrenergic receptor comprises a glucocorticosteroid. In some embodiments, the treatment region is defined by application of a temporary tattoo or stencil to the treatment region; provided that the temporary tattoo or stencil is configured to facilitate the administration of the composition to distinct areas of the region; and provided that the tattoo or stencil indicates from about 1 to about 100 distinct areas.

In some embodiments, the composition is administered to the treatment region in one or more sessions, wherein for each session the composition is administered in a total dosage comprising from about 1 nanogram to about 10 micrograms beta-2 adrenergic receptor agonist, and the total dosage is distributed over about 1 to about 100 distinct areas of the region during each session. In some embodiments, from about 0.004 micrograms to about 0.02 micrograms of beta-2 adrenergic receptor agonist is administered to one or more of the distinct areas of the treatment region during each session. In some embodiments, the distance between two adjacent distinct areas of the region is from about 0.5 cm to about 10. In some embodiments, the treatment region comprises a submental region, central abdominal region, face, flank, back, chest, arm, leg, buttock, or combination thereof. In some embodiments, the treatment region is a central abdominal region comprising less than about 600 cm² subcutaneous fat around the umbilicus; a submental region comprising less than about 400 cm² subcutaneous fat; or a combination thereof. In some embodiments, the treatment region is a central abdominal region and the distance between two adjacent distinct areas of the region is about 4 cm. In some embodiments, the treatment region is a central abdominal region and the composition is administered between axial planes located at about +70 mm and about −90 mm relative to the umbilicus. In some embodiments, the treatment region is a submental region and the distance between two adjacent distinct areas of the region is about 1 cm. In some embodiments, the treatment region is a submental region and 0.02 micrograms per milliliter of beta-2 adrenergic receptor agonist is administered to the region in 0.2 ml injections.

In another aspect of the subject matter described herein, provided is a method of determining an effect of a body contouring treatment to a treatment region of a patient's body, the method comprising performing one or more assessments after administration of a composition comprising an effective amount of a long-acting beta-2 adrenergic receptor agonist to the treatment region, provided that the treatment region comprises subcutaneous adipose tissue or bulging due to subcutaneous adipose tissue prior to administration and provided that the patient is non-obese. In some embodiments, the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, or a salt, solvate, polymorph, or combination thereof. In some embodiments, the beta-2 adrenergic receptor agonist comprises salmeterol xinafoate. In some embodiments, the composition further comprises one or more compounds to prevent or reduce desensitization of f3-adrenergic receptors, an antihistamine, or a combination thereof. In some embodiments, the one or more compounds to prevent or reduce desensitization of β-adrenergic receptors comprises a glucocorticosteroid. In some embodiments, the treatment region comprises a submental region, central abdominal region, face, flank, back, chest, arm, leg, buttock, or combination thereof.

In some embodiments, the one or more assessments comprises evaluating visible fat protrusion or bulging in the treatment region by selecting a descriptive phrase to describe the visible fat protrusion or bulging from an assessment scale comprising from about 3 to about 10 descriptive phrases that each indicate varying degrees of visible fat protrusion or bulging. In some embodiments, each descriptive phrase of the assessment scale is converted to a number on an ordinal scale to generate an assessment score; and provided that the lowest assessment score indicates no or a low level of visible fat protrusion or bulging and the highest assessment score indicates a high level of visible fat protrusion or bulging. In some embodiments, the evaluation is performed both prior to and after the body contouring treatment and provided that the body contouring treatment is effective if the assessment score after the treatment is lower than the assessment score prior to treatment.

In some embodiments, the one or more assessments comprises performing a match-to-sample task comprising rating visible fat protrusion in the treatment region of the patient on a photonumeric scale comprising from about 3 to about 10 representative pictures of increasing visible fat protrusion or bulging in corresponding reference treatment regions, each picture having a numeric value directly proportional to amount of visible fat protrusion or bulging. In some embodiments, the match-to-sample task is performed both prior to and after the body contouring treatment and provided that the body contouring treatment is effective if the numeric value from the photonumeric scale after treatment is at least 1 or 2 values lower than the numeric value from the photonumeric scale prior to treatment.

In some embodiments, the one or more assessments comprises measuring the treatment region using a laser-guided manual tape measure; three-dimensional digital photographic imaging of the treatment region; measuring subcutaneous fat of the treatment region using a skin-fold caliper; subjective photographic assessment of the treatment region; administering one or more questionnaires to the patient, a clinician, or both the patient and clinician; performing magnetic resonance imaging of the treatment region; performing ultrasound imaging of the treatment region; visual grading; palpating the treatment region; or a combination thereof. In some embodiments, the treatment region comprises a visible protrusion of subcutaneous adipose tissue or bulging prior to administration of the compositions; provided that the one or more assessments comprises measuring the visible protrusion or bulging using a laser-guided manual tape measure, a skin-fold caliper, or a combination thereof; and provided that after administration of the composition, the protrusion or bulging decreases by at least about 0.5, 1 or 2 cm. In some embodiments, the one or more assessments comprises measuring the treatment region by magnetic resonance imaging and/or ultrasound, and provided that after administration of the composition, subcutaneous fat of the treatment region decreases by at least about 1 mm, 2 mm, 3 mm, 4 mm, 5 mm, 10 mm, 20 mm or more.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic illustrating a putative reduction of an adipose cell following administration of a composition comprising a beta-2 adrenergic receptor agonist.

FIGS. 2A-B are graphical illustrations of the submental regions of a female patient (2A) and a male patient (2B) that could be suitable for treatment with a composition comprising a beta-2 adrenergic receptor agonist (panel 1), with possible post-treatment reduction shown (panel 2).

FIGS. 3A-B are graphical illustrations of the central abdominal regions of a female patient (3A) and male patient (3B) that could be suitable for treatment with a composition comprising a beta-2 adrenergic receptor agonist (panel 1), with possible post-treatment reduction shown in panel 2 and an overlay of the before and after treatment illustrations shown in panel 3.

FIG. 4 illustrates an embodiment of a tattoo or stencil useful for the administration of a composition to a treatment region of the body.

FIG. 5 illustrates a second embodiment of a tattoo or stencil useful for the administration of a composition to a treatment region of the body.

FIG. 6 illustrates a third embodiment of a tattoo or stencil useful for the administration of a composition to a treatment region of the body.

FIG. 7 illustrates a fourth embodiment of a tattoo or stencil useful for the administration of a composition to a treatment region of the body.

FIGS. 8A-B illustrate six representative pictures of the abdomens of male subjects (8A) and female subjects (8B) having various degrees of central abdominal bulging.

FIG. 9 illustrates the measurement of the central abdominal region of a patient using a laser-guided manual tape measure.

FIG. 10 is a graphical representation of the reductions in abdominal circumference at the umbilicus (panel A) and volume in the treatment region (panel B) after administration of a composition comprising salmeterol xinafoate compared to placebo.

FIG. 11 is a graphical representation of the effects of weight change on reductions in abdominal circumference at the umbilicus (panel A) and volume in the treatment region (panel B) after administration of a composition comprising salmeterol xinafoate compared to placebo.

FIG. 12 is a graphical representation of the increase in the percentage of composite 1-point P-GAPS and 2-point CPnS responders (panel A) and increase in the percentage of composite 2-point P-GAPS and 2-point CPnS responders (panel B) after administration of salmeterol xinafoate compared to placebo.

FIGS. 13A-B exemplify two responders to salmeterol xinafoate treatment. FIG. 13A shows photographs of a 37 year old female, BMI=25.8 kg/m2, treated with 0.4 μg salmeterol xinafoate at screening (panel A) and End-of-Study, day 57 (panel B). FIG. 13B shows photographs of a 30 year old male, BMI=24.9 kg/m2, treated with 4.0 μg salmeterol xinafoate at screening (panel A) and End-of-Study, day 57 (panel B).

### DETAILED DESCRIPTION OF THE INVENTION

Adipose tissue is the primary energy storage tissue of the body. Fat cells, or adipocytes, store this energy in the form of triglycerides. Triglycerides are mobilized from fat stores to provide caloric energy to the body through hormonal induction of triglyceride hydrolysis. This process releases free or non-esterified fatty acids and glycerol into the blood for use by other body tissues. The breakdown of triglycerides from fat store is referred to as lipolysis. Growth of new adipocytes also occurs, which is referred to as adipogenesis. Two of the neurotransmitters that control lipolysis in the body are the catecholamines epinephrine and norepinephrine. Adipose tissue has beta-1, -2, and -3 adrenergic receptors and alpha-2 adrenergic receptors. Binding of beta-adrenergic receptor agonists (“beta-adrenergic agonists”) to beta-adrenergic (“beta”) receptors in adipose tissue results in lipolysis within adipocytes. Beta-adrenergic receptor activation also inhibits adipogenesis. In humans, beta-2 receptors are the most abundant on fat cell surfaces and the primary mediator of beta-adrenergic receptor-stimulated lipolysis. Stimulation of lipolysis by beta-adrenergic agonists is mediated by adenylate cyclase and increased formation of cyclic adenosine monophosphate (cyclic AMP, cAMP).

Long-acting beta-2 adrenergic receptor agonists, such as salmeterol and formoterol, reduce regional fat deposits or adipose tissue regions by binding to beta receptors, resulting in adipocyte lipolysis. The systemic use of beta-2 adrenergic receptor agonists, however, carries with it possible side effects that are potentially life-threatening. For example, use of long-acting beta-2 adrenergic receptor agonists may result in cardiovascular problems such as angina, hypertension or hypotension, tachycardia, palpitations, and arrhythmias. Thus, while long-acting beta-2 adrenergic receptor agonists may reduce regional fat deposits and adipose tissue regions, they may also cause increased heart rate and palpitations. A benefit of regional, low-dosage administration is the avoidance of serious side effects associated with systemic administration.

Combined with appropriately administered amounts via subcutaneous injection, certain lipophilic long-acting beta-2 adrenergic receptor agonists provide, in many instances, therapeutic and/or cosmetic effectiveness in body contouring applications by reducing regional fat deposits and/or adipose tissue. This reduction in regional fat deposits and/or adipose tissue in select body regions has been evaluated in patients identified as suitable for body contouring treatment.

In one aspect, provided herein are methods of treatment for body contouring comprising the administration to a patient a composition comprising a beta-2 adrenergic agonist to a region of the body, provided that the patient has been identified as suitable for body contouring treatment as determined by one or more assessments. Beta-2 adrenergic agonists include, without limitation, salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, and salts, solvates, polymorphs, or combinations thereof. Assessments include the determination of the presence of subcutaneous fat in the treatment region of the body. The treatment region, in various embodiments, includes regions of the body having visible and protruding amounts of subcutaneous fat, including, without limitation, head and neck (including face), flank, abdomen, back, chest, arm, leg and buttock. The head and neck region includes, without limitation, orbit, periorbit, neck, chin and submental region. A patient having submental fat, in many instances, is suitable for body contouring treatment, wherein the treatment is administered to the submental region. The visible and protruding amounts of subcutaneous fat often presents as bulging. An example of bulging is central abdominal bulging, which is often visually apparent in a patient. A patient having central abdominal bulging due to subcutaneous fat, in many instances, is suitable for body contouring treatment, wherein the treatment is administered to the central abdominal region. A patient suitable for body contouring treatment, in some embodiments, is non-obese. In some embodiments, a patient is determined to be non-obese by measuring their body mass index (BMI). In some instances, a patient with a BMI less than about 30 kg/m² is non-obese.

In one aspect, provided herein are methods for determining patient suitability for body contouring treatment administered to a region of the body, by performing one or more assessments on the patient. The patient selection assessments include those previously described, for example, determining if a patient is non-obese and/or determining if a patient has subcutaneous fat in the region of the body of treatment interest.

In one aspect, one or more assessments performed prior to a body contouring treatment are also performed after said treatment to determine efficacy of the treatment. In some embodiments, an assessment comprises a clinician and/or patient rating the body contour (e.g., visible protruding fat or bulging) of the desired treatment region on a descriptive and/or numeric scale. In some examples, an assessment of submental fat comprises a clinician and/or patient rating the contour of the submental region on a scale. In some examples, an assessment of a central abdominal region comprises a clinician and/or patient rating the level of bulging on a scale. In some instances, the scale is a photonumeric scale. In some embodiments, the assessment is performed prior to and after the administration of a composition comprising a beta-2 adrenergic receptor agonist to the treatment region.

### Patient Selection

In one aspect, a patient suitable for body contouring treatment (e.g., localized/regional fat reduction) has subcutaneous fat in the region of the body targeted for treatment. Body contouring treatments comprise administration of a composition comprising a beta-2 adrenergic receptor agonist to a region of the body of interest. In many instances, the beta-2 adrenergic receptor agonist is a long-acting beta-2 adrenergic receptor agonist. In some embodiments, the beta-2 adrenergic receptor agonist comprises salmeterol, or a salt or solvate of salmeterol, or a combination thereof. In some embodiments, the beta-2 adrenergic receptor agonist comprises salmeterol xinafoate.

One common region comprising subcutaneous fat is the central abdominal area. The subcutaneous fat in this region, in many circumstances, is referred to as central abdominal bulging. In some embodiments, central abdominal bulging is descriptive of subcutaneous fat in the central abdominal region that is often characterized as rolls, pooch, spare tire, bloat, paunch, love-handles, pot-belly, pooch, stomach rolls, among a number of other commonly used terms. In addition to its visual appearance, the area may be described as feeling flabby or jiggly and the appearance of the area as unattractive or ugly. In some patients, concerns over physical appearance or body image regarding the bulging leads to impairment in self-image, confidence and overall daily functioning due to social inhibition and anxiety. Consequently, these patients, in many instances, are suitable for body contouring treatment to achieve a reduced level of bulging, for example, a flatter abdomen. In many cases, compositions provided herein comprising a beta-2 adrenergic receptor agonist administered to the central abdominal region have a statistically significant reduction in central abdominal bulging due to subcutaneous fat following a treatment regimen. In particular, patients experiencing a reduction in central abdominal bulging due to subcutaneous fat are non-obese patients.

Another region that often comprises troublesome subcutaneous fat in some patient populations is the submental region. The subcutaneous fat in this region, in many circumstances, is referred to as submental fat. In some embodiments, submental fat is descriptive of subcutaneous fat in the submental region that is often characterized as submental fold, double chin, turkey neck, sagging neck, dewlap, wattle, among a number of other commonly used terms. In addition to its visual appearance, the area may be described as feeling flabby or jiggly and the appearance of the area as unattractive or ugly. In some patients, concerns over physical appearance or body image regarding the fat leads to impairment in self-image, confidence and overall daily functioning due to social inhibition and anxiety. Consequently, these patients, in many instances, are suitable for body contouring treatment to achieve a reduced level of subcutaneous fat or a reduced visual appearance of fat, for example, a thinner neck. Compositions provided herein comprising a beta-2 adrenergic receptor agonist administered to the submental region, in various embodiments, have a statistically significant reduction in subcutaneous fat or the visual appearance of fat following a treatment regimen. In particular, patients experiencing a reduction in subcutaneous fat or the visual appearance of fat are non-obese patients.

In one aspect, a patient suitable for body contouring treatment is evaluated for central abdominal bulging treatment by one or more established diagnostic criteria identifying (diagnosing) a patient as having central abdominal bulging. Central abdominal bulging due to subcutaneous fat, in many instances, presents as periumbilical bulging, or bulging around the navel, due to an accumulation of excessive subcutaneous fat. One of the hallmarks of this condition includes focal periumbilical bulging, where localized subcutaneous fat in the central abdomen in a patient, often a non-obese patient, is clinically apparent as a distinctly visible and palpable area of periumbilical soft tissue bulging, often flanked by flat or concave lateral areas. A palpation assessment to diagnose palpable periumbilical subcutaneous fat in the central abdominal region is often made while a patient assumes proper posture, does not contract their abdomen and the assessment is made on full, natural exhalation. This palpable periumbilical subcutaneous fat is often up to approximately 8 cm in patients who are non-obese. In some embodiments, a pinch test is useful for estimating skin-fold thickness between the thumb and forefinger(s) for the presence of subcutaneous fat retractable from the abdominal musculature and not the result of visceral fat, which confirms the diagnosis of subcutaneous fat in the central abdomen. A patient suitable for localized fat reduction in the central abdomen, in many instances, has less than about 8 cm of subcutaneous fat as measured by the pinch test described above, a caliper or ultrasound. In some embodiments, a patient suitable for localized fat reduction in the central abdomen has less than about 15 cm, 14 cm, 13 cm, 12 cm, 11 cm, 10 cm, 9 cm, 8 cm, 7 cm, 6 cm, 5 cm, 4 cm, 3 cm or 2 cm of subcutaneous fat in the central abdominal region. Another hallmark of central abdominal bulging is absence of other abnormalities such as rectus diastasis, hernias or any musculo skeletal abnormalities that could account for the periumbilical bulging. In many instances, central abdominal bulging presents in individuals, often non-obese individuals, as periumbilical bulging due to an accumulation of excessive subcutaneous fat that is often unresponsive to diet and exercise.

In one aspect, a patient suitable for body contouring treatment is evaluated for submental treatment by one or more established diagnostic criteria identifying (diagnosing) a patient as having submental fat. Submental fat comprising excess subcutaneous fat, in many instances, presents as a visual protrusion of fat and/or skin around the neck, due to an accumulation of excessive subcutaneous fat. One of the hallmarks of this condition includes a clinically apparent, distinctly visible and palpable area of soft tissue (e.g., skin, fat, skin and fat) protrusion. A palpation assessment to diagnose palpable submental subcutaneous fat is often made while a patient assumes proper posture. This palpable submental subcutaneous fat is often up to approximately 5 cm in patients who are non-obese. In some embodiments, a pinch test is useful for estimating skin-fold thickness between the thumb and forefinger(s) for the presence of subcutaneous fat retractable from the neck and not the result of visceral fat, which confirms the diagnosis of subcutaneous fat in the submental region. A patient suitable for localized fat reduction in the submental region, in many instances, has less than about 5 cm of subcutaneous fat as measured by the pinch test described above, a caliper or ultrasound. In some embodiments, a patient suitable for localized fat reduction in the submental region has less than about 10 cm, 9 cm, 8 cm, 7 cm, 6 cm, 5 cm, 4 cm, 3 cm, 2 cm, 1.5 cm, 1 cm, or 0.5 cm of subcutaneous fat in the submental region. Another hallmark of submental fat is absence of other abnormalities such as musculoskeletal abnormalities that could account for the protrusion of soft tissue. In many instances, submental fat presents in individuals, often non-obese individuals, as a visual protrusion due to an accumulation of excessive subcutaneous fat that is often unresponsive to diet and exercise.

In one aspect, a patient suitable for body contouring treatment is non-obese. One measure to determine if a patient is non-obese is to measure the patient's body mass index (BMI). A patient with a BMI greater than or equal to 30 kg/m², in many instances, is obese. BMI is calculated from a determination of weight measured in kilograms and height measured in meters. In some embodiments, a patient with a BMI greater than or equal to 30 kg/m² may have, as an example, a greater amount of body mass due to muscle than subcutaneous fat. Such patients may be an exception and are suitable patients for body contouring treatment. In some instances, a patient suitable for body contouring treatment is obese, for example, a patient suitable for body contouring treatment has a BMI of about 30, 31, 32, 33, 34, 35, 36, 37, 38, 39 or 40 kg/m². In some embodiments, a patient with central abdominal bulging is not obese, for example, the patient has a BMI less than or equal to 30 kg/m². In some embodiments, a patient with submental fat is not obese, for example, the patient has a BMI less than or equal to 30 kg/m². In exemplary embodiments, a patient suitable for body contouring treatment has a BMI of about 18, 19, 20, 21, 22, 23, 24, 25, 26, 27, 28 or 29 kg/m². In many circumstances, a patient suitable for body contouring treatment does not have a BMI less than about 18 kg/m². Body contouring treatment includes administration of a composition comprising a beta-2 adrenergic receptor agonist to a region of the body of interest. In many instances, the beta-2 adrenergic receptor agonist is a long-acting beta-2 adrenergic receptor agonist. In some embodiments, the beta-2 adrenergic receptor agonist comprises salmeterol, or a salt or solvate of salmeterol, or a combination thereof. In some embodiments, the beta-2 adrenergic receptor agonist comprises salmeterol xinafoate.

In one aspect, a patient suitable for body contouring treatment has a treatment region comprising from about 1 cm to about 20 cm of subcutaneous fat thickness, as measured using any method described herein including, but not limited to, use of a caliper, pinch test or ultrasound. A treatment region includes, without limitation, face, flank, back, neck, chin, submental, facial, arm, leg, waist, chest, hip and buttock. The measurement of subcutaneous fat thickness may be performed at any area of the region. For example, for measuring the central abdominal region, measurement areas include, without limitation, the waist, abdomen, central abdomen, umbilicus, periumbilicus, and oblique area. Fat thickness and/or volume, in many instances, varies depending on the area of the measurement region. For example, fat thickness at the oblique area may differ from fat thickness at the umbilicus. Wherein the region is the central abdominal region, in many instances the patient is suitable for treatment if the region comprises less than about 20 cm, less than about 19 cm, less than about 18 cm, less than about 17 cm, less than about 16 cm, less than about 15 cm, less than about 14 cm, less than about 13 cm, less than about 12 cm, less than about 11 cm, less than about 10 cm, less than about 9 cm, less than about 8 cm, less than about 7 cm, less than about 6 cm, less than about 5 cm, less than about 4 cm, less than about 3 cm, less than about 2 cm or less than about 1 cm subcutaneous fat. In some embodiments, a patient suitable for central abdominal body contouring treatment has from about 1 cm to about 12 cm, from about 1 cm to about 11 cm, from about 1 cm to about 10 cm, from about 1 cm to about 9 cm, from about 1 cm to about 8 cm, from about 1 cm to about 7 cm, from about 1 cm to about 6 cm, from about 1 cm to about 5 cm, from about 1 cm to about 4 cm, from about 1 cm to about 3 cm, from about 1 cm to about 2, from about 2 cm to about 10 cm, from about 2 cm to about 9 cm, from about 2 cm to about 8 or from about 2 cm to about 7 cm of subcutaneous fat in the central abdominal region. The measurement of subcutaneous fat may be performed on any region of the central abdominal region, for example, at or around the umbilicus. Wherein the region is the submental region, in many instances the patient is suitable for treatment if the region comprises less than about 10 cm, less than about 9 cm, less than about 8 cm, less than about 7 cm, less than about 6 cm, less than about 5 cm, less than about 4 cm, less than about 3 cm, less than about 2 cm or less than about 1 cm subcutaneous fat. In some embodiments, a patient suitable for submental body contouring treatment has from about 0.5 cm to about 10 cm, from about 0.5 cm to about 9 cm, from about 0.5 cm to about 8 cm, from about 0.5 cm to about 7 cm, from about 0.5 cm to about 6 cm, from about 0.5 cm to about 5 cm, from about 0.5 cm to about 4 cm, from about 0.5 cm to about 3 cm, from about 0.5 cm to about 2, from about 1 cm to about 10 cm, from about 1 cm to about 9 cm, from about 1 cm to about 8 or from about 1 cm to about 7 cm of subcutaneous fat in the submental region. The measurement of subcutaneous fat may be performed on any region of the submental region, for example, at or around the submental triangle.

In one aspect, a patient suitable for body contouring treatment is assessed using a tape measure procedure. In some cases, the tape measurement is guided using a laser. In some embodiments, a patient suitable for body contouring treatment is assessed using a constant-tension tape measure. In some embodiments, a patient suitable for body contouring treatment is assessed using a skin-pinch caliper. In some embodiments, a patient suitable for body contouring treatment is assessed using ultrasound.

In one aspect, a patient suitable for body contouring treatment expresses dissatisfaction with the contour of a region of the body targeted for treatment. A patient suitable for body contouring treatment may have any number of reasons for selecting treatment, not limited to, a desire to maintain an “ideal” physical appearance (e.g., lean, symmetrical body image). As an exemplary embodiment, a patient suitable for treatment is dissatisfied with the contour of their central abdomen. As another exemplary embodiment, a patient suitable for treatment is dissatisfied with the contour of their submental region.

In one aspect, a patient self-assessment is performed to determine suitability for body contouring treatment. In another or additional aspect, a clinician assessment is performed to determine patient suitability for body contouring treatment. In some embodiments, a patient and/or clinician assessment comprises a match-to-sample task, wherein the clinician and/or patient rate the contour and/or visual appearance of the treatment region on a photonumeric scale. In some embodiments, a patient and/or clinician assessment comprises a match-to-sample task, wherein the clinician and/or patient rate the amount of bulging in the region of the patient on a photonumeric scale. In some embodiments, an assessment comprises obtaining measurements of the treatment region. Measurements include the direct and indirect measure of subcutaneous fat volume, fat thickness, and/or fat circumference. Measurements may be performed using any suitable device known to one in the art, including, without limitation, tape measure, caliper, magnetic resonance imaging and ultrasound.

In some instances, a patient is determined to be suitable for body contouring treatment by evaluating a patient self-assessment. In some instances, a patient is determined to be suitable by evaluating a clinician assessment. In some instances, a patient is determined to be suitable by analysis of both patient and clinician reported assessments. In various embodiments, a patient is determined suitable for body contouring treatment by any combination of assessments performed by the patient, clinician or both the patient and clinician. A clinician includes, without limitation, a dermatologist, plastic surgeon, primary care physician, OB/GYN, members of other specialties, some of whom perform liposuction, non-invasive fat reduction and other procedures for fat reduction, registered nurse, physician assistant and any physician with a medical degree such as a M.D. or D.O. In many instances, the clinician who performs a patient assessment is the same clinician to administer treatment. In some instances, a clinician who performs a patient evaluation is a different person from the clinician who administers treatment. In some embodiments, the assessment and administration are performed by different clinicians at different locations.

In various embodiments, a patient suitable for body contouring treatment has been evaluated with a physical examination and/or standard history prior to proceeding to a consideration of body contouring treatment.

### Compositions

In one aspect, provided herein are compositions comprising beta-2 adrenergic receptor agonists for administration to a patient suitable for body contouring treatment, wherein the body contouring treatment functions to reduce regional fat deposits, reduce adipose tissue and/or reduce the visual appearance of excess subcutaneous fat. In some embodiments, the beta-2 adrenergic receptor agonists are long-acting. In some embodiments, the beta-2 adrenergic receptors are lipophilic.

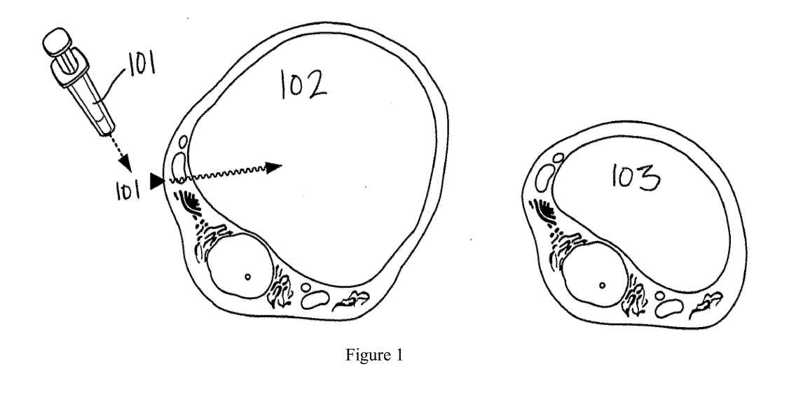

Adrenergic receptors play a major role in the regulation of several processes in the body, including fat cell metabolism. As shown in FIG. 1, an adrenergic receptor agonist, 101, such as salmeterol xinafoate, activates beta-2 adrenergic receptors located on human fat cells and triggers the metabolism of triglycerides in these cells to free fatty acids and glycerol by means of lipolysis. FIG. 1 exemplifies this process by showing the injection of a beta-2 adrenergic receptor agonist to a region of the body, wherein the agonist, 101, is delivered to a fat cell, 102, to reduce cell size, 103. Administration of the composition evenly across a region of the body, such as the abdomen and/or submental region, in many instances, has the effect of shrinking fat cells uniformly to reduce the visual protrusion or bulging of subcutaneous fat. In some embodiments, the administration of the composition does not result in contour irregularities. In some circumstances, unlike other treatments which remove, damage or kill fat cells, the administration of a composition provided herein functions to reduce local fat stores and the bulges or visual protrusions they create, often with no inflammatory reaction. The composition functions, in many circumstances, to reduce fat tissue in selective regions without ablation or surgery, providing a type of pharmaceutical lipoplasty.

In some embodiments, sustained modulation of adrenergic receptors in adipose tissue result in some combination of sustained lipolysis, reduced lipid content of the adipocyte, reduced adipocyte cell size, reduced adipose tissue mass or fat accumulation, and/or improved cosmetic appearance. Some embodiments provide selective reduction of regional and/or subcutaneous accumulations of adipose tissue and adipocytes, including cellulite, through sustained adrenergic modulation. Sustained adrenergic modulation results in sustained inhibition of fat cell proliferation (adipogenesis) in some embodiments. In some embodiments, the composition is useful for treating cellulitic fat accumulation and/or lipomas.

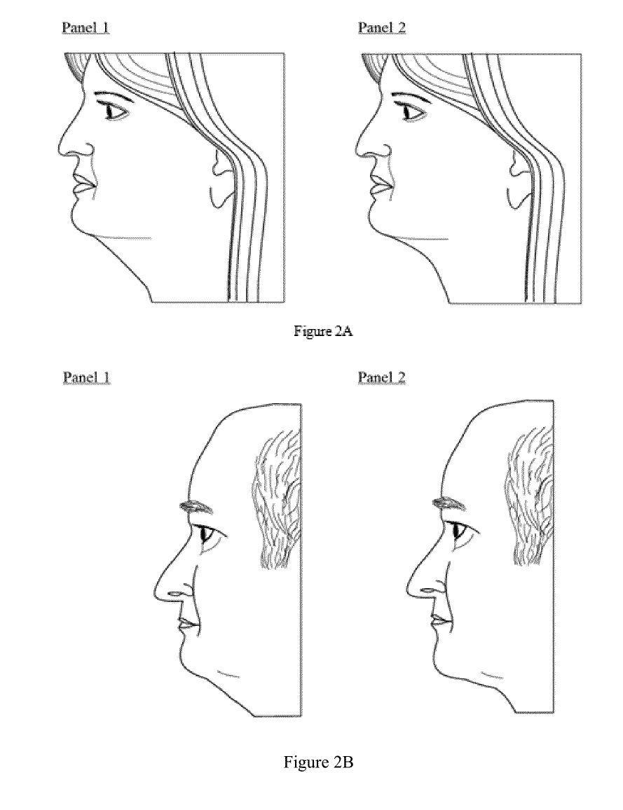

A composition comprising a beta-2 adrenergic receptor agonist, in various embodiments, is suitable for administration to a submental region of an individual to reduce subcutaneous fat in said region. Exemplary patients suitable for submental body contouring treatment are shown in panel 1 of FIGS. 2A and 2B. A visual reduction of submental fat is often achieved, as simulated in panel 2 of FIGS. 2A and 2B after a treatment regimen comprising the administration of a beta-2 adrenergic receptor agonist to the submental region. The top panel of FIG. 2 represents a submental region of a female patient prior to and after treatment with a composition comprising a beta-2 adrenergic receptor agonist. The bottom panel of FIG. 2 represents a submental region of a male patient prior to and after treatment with a composition comprising a beta-2 adrenergic receptor agonist.

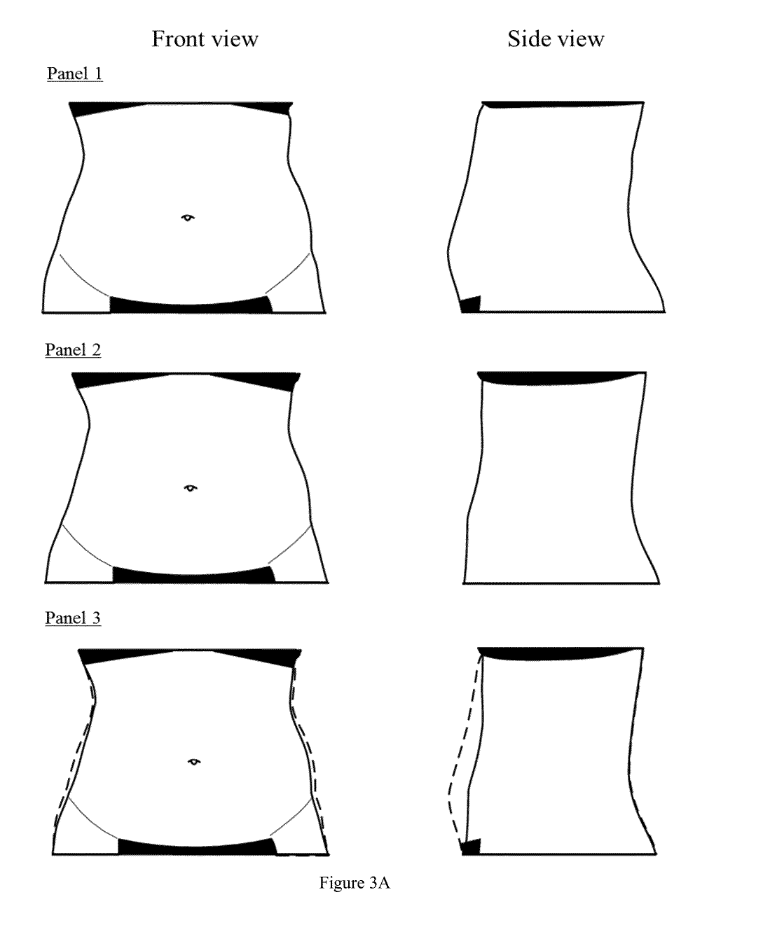

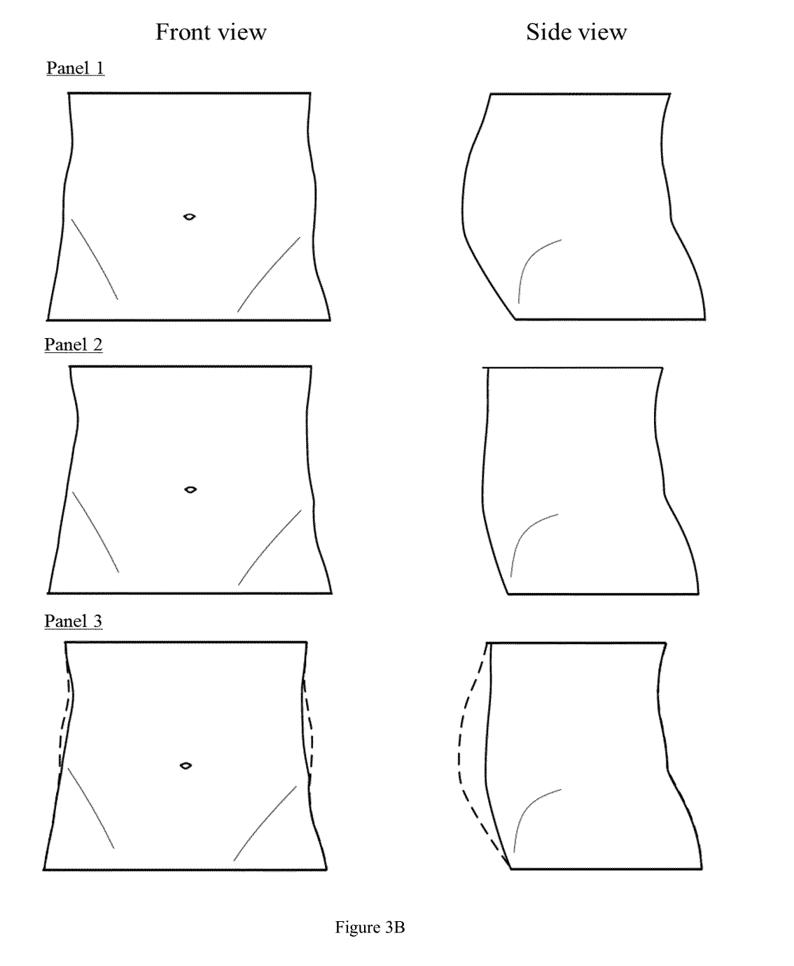

A composition comprising a beta-2 adrenergic receptor agonist, in various embodiments, is suitable for administration to a central abdominal region of an individual to reduce subcutaneous fat in said region. A visual reduction is often achieved, as simulated in FIGS. 3A-3B. FIG. 3A represents a central abdominal region of a female patient prior to (panel 1) and after treatment (panel 2) with a composition comprising a beta-2 adrenergic receptor agonist. FIG. 3B represents a central abdominal region of a male patient prior to and after treatment with a composition comprising a beta-2 adrenergic receptor agonist. The top panels of each figure represent the individual prior to a treatment. The central panels represent the individual after a treatment, for example, a treatment comprising the administration of a composition comprising salmeterol xinafoate, once weekly, for a period of 8 weeks. Panel 3 of each figure illustrate an overlap between the top and central panels, wherein the dashed line is indicative of the abdomen prior to treatment and the solid line is indicative of the abdomen after treatment.

The composition, in various embodiments, does not have the typical risks and adverse events seen with current surgical and non-surgical options. In some embodiments, the safety profile of the composition comprising a beta-2 adrenergic receptor agonist is similar to placebo, such as sodium chloride injection. Reactions which may occur after administration of the composition by injection, are in many instances infrequent and similar to those of placebo injections, such as transient injection site reactions, such as erythema, hematoma and pain. These reactions, in many instances, have been shown to be infrequent and at the same rate as placebo injections, suggesting that these adverse events are related to the injection procedure itself and not the composition. In some embodiments, the patient is not anesthetized before or during administration of the composition. In some embodiments, the composition does not result in an inflammatory reaction.

The composition for body contouring treatment provided herein comprises a beta-2 adrenergic receptor agonist. In some embodiments, the beta-2 adrenergic receptor agonist is long-acting. In some embodiments, the beta-2 adrenergic receptor agonist is lipophilic. In some embodiments, the beta-2 adrenergic receptor agonist comprises salmeterol, a salt or solvate thereof, or a combination thereof. In some embodiments, the beta-2 adrenergic receptor agonist comprises salmeterol xinafoate. In some embodiments, the long-acting beta-2 adrenergic receptor agonist is selected from salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof. Long-acting beta-2 adrenergic receptor agonists include ultra long-acting beta-2 adrenergic receptor agonists. In some embodiments, the beta-2 adrenergic receptor agonist is a dual agonist, for example, sibenadet. In some embodiments, the composition comprises a short-acting beta-2 adrenergic receptor agonist. Short-acting beta-2 adrenergic receptor agonists include, without limitation, albuterol, levalbuterol, terbutaline, pirbuterol, procaterol, clenbuterol, metaproterenol, fenoterol, bitolterol, ritodrine, salbutamol, and isoprenaline. Exemplary short-acting beta-2 adrenergic receptor agonists include, without limitation, albuterol sulfate, levalbuterol HCl and bitolterol mesylate. Beta-2 adrenergic receptor agonists include, without limitation, bromoacetylalprenololmenthane, broxaterol, cimaterol, dopexamine, hexoprenaline, higenamine, isoxsuprine, isoproterenol, mabuterol, methoxyphenamine, nylidrin, primidolol, ractopamine, reproterol, rimiterol, tretoquinol, tulobuterol, zilpaterol, zinterol, orciprenaline, butaxamine, pindolol, dobutamine, mirabegron, isoetharine, carbuterol, clorprenaline, colterol, denopamine, dioxethedrine, ephedrine, etafedrine, ethylnorepinephrine, ibopamine, ibuterol, levabuterol, oxyfedrine, picumeterol, prenalterol, protokylol, soterenol, tetoquinol, and xamoterol.

The composition for body contouring treatment, in various embodiments, comprises a combination of a beta-2 adrenergic receptor agonist (e.g., salmeterol xinafoate) and a glucocorticoid, such as fluticasone propionate. Glucocorticoids, like fluticasone propionate, potentially enhance the activity of the beta-2 adrenergic receptor agonist, such as salmeterol. In some instances, this composition is administered to the orbit of a patient having symptomatic exophthalmos, or protrusion of the eye from the orbit associated with thyroid-related eye disease. Exophthalmos associated with thyroid-related eye disease is caused by expansion of fat and muscle behind the eye.

In some embodiments, the composition selectively partitions into adipose tissue relative to blood plasma after administration. It has been found that certain lipophilic long-acting selective beta-2 adrenergic agonists administered subcutaneously in appropriate amounts have limited systemic exposure compared to other long-acting beta-2 adrenergic agonists. One possible reason for this result is that the lipophilic nature of certain long-acting beta-2 adrenergic receptor agonists allows selective partitioning into the adipose tissue relative to plasma. In many instances, the lipophilicity of certain long-acting beta-2 adrenergic receptor agonist into the subcutaneous adipose tissue contributes, at least in part, to providing relatively low levels of the agonist systemically. Appropriate amounts of the lipophilic long-acting beta-2 adrenergic receptor agonists described herein administered via subcutaneous or transcutaneous injection may provide therapeutic effectiveness in reducing regional fat deposits and/or adipose tissue with a reduced risk of producing systemic side effects. The composition provided herein, in many embodiments, is injectable. The composition is suitable for subcutaneous administration. In further or additional embodiments, the composition is suitable for transcutaneous administration. In some embodiments, the composition is formulated for transdermal administration. Accordingly, the compositions and methods of treatment described herein are designed to reduce subcutaneous adipose tissue which is distinct from visceral fat.

Salmeterol, an exemplified beta-2 adrenergic receptor agonist of a composition provided herein, has high lipid solubility which extends its residence time in the adipose tissue and/or in one or more adipose cells. Some embodiments of the composition comprise a highly lipophilic beta-adrenergic agonist, which reduces or eliminates the need for a sustained or controlled release carrier due to partitioning and sequestration in the adipose tissue, thereby prolonging the treatment effect. In some embodiments, lipophilic beta-adrenergic agonists with an oil-water partition coefficient of at least about 1,000 or at least about 10,000 to 1 are used. For example, salmeterol is at least 10,000 times more lipophilic than albuterol, a short-acting hydrophilic beta-adrenergic agonist.

In some embodiments, the composition comprises an adipose tissue-reducing amount of salmeterol or a salt, optical isomer, racemate, solvate, or polymorph thereof. In some embodiments, the composition further comprises at least one subcutaneously acceptable inactive ingredient, wherein the composition provides a salmeterol plasma Cmax ratio of subcutaneous to intravenous administration of between about 0.01 to about 0.4 when administered subcutaneously (also known as the “salmeterol partition ratio”). In one embodiment, the partition ratio of salmeterol is about 0.01 to about 0.4. In another embodiment, the salmeterol partition ratio is about 0.05 to about 0.3. In another embodiment, the salmeterol partition ratio is from about 0.1 to about 0.35. In a further embodiment, the salmeterol partition ratio is about 0.1. In another embodiment, the salmeterol partition ratio is between 0.05 to about 0.2. In a further embodiment, the salmeterol partition ratio is between about 0.1 to about 0.2. In yet another embodiment, the salmeterol partition ratio is about 0.01, about 0.02, about 0.03, about 0.04, about 0.05, about 0.06, about 0.07, about 0.08, about 0.09, about 0.1, about 0.11, about 0.12, about 0.13, about 0.14, about 0.015, about 0.16, about 0.17, about 0.18, about 0.19, about 0.2, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40. As used herein, in many instances, a salmeterol-like compound is a compound having a partition ratio of between about 0.01 and 0.4 and provides limited systemic exposure, and consequently, a reduced or minimized risk of producing systemic side effects. Additionally, salmeterol-like compounds also selectively partition into the adipose tissue due to their lipophilic nature.

Disclosed herein are compositions comprising a beta-2 adrenergic receptor agonist. The compositions may comprise 1, 2, 3, 4, or more beta-2 adrenergic receptor agonists. The beta-2 adrenergic receptor agonists, in some embodiments, comprise salts, solvates, or a combination thereof. The compositions, in various embodiments, further comprise one or more pharmaceutically acceptable salts, excipients or vehicles. Pharmaceutically acceptable salts, excipients, or vehicles include carriers, excipients, diluents, antioxidants, preservatives, coloring, diluting agents, emulsifying agents, suspending agents, solvents, fillers, bulking agents, buffers, delivery vehicles, tonicity agents, cosolvents, complexing agents, antimicrobials, and surfactants. Neutral buffered saline or saline mixed with serum albumin are exemplary appropriate carriers. The composition may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Suitable preservatives include benzalkonium chloride, thimerosal, phenethyl alcohol, methylparaben, propylparaben, chlorhexidine, sorbic acid and the like. Hydrogen peroxide is also sometimes used as a preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Buffers include conventional buffers such as acetate, borate, citrate, phosphate, bicarbonate and Tris-HCl.

In some embodiments, the composition is in liquid form or in a lyophilized or freeze-dried form which in some instances includes one or more lyoprotectants, excipients, surfactants, high molecular weight structural additives and/or bulking agents. In one embodiment, a lyoprotectant is included, which is a non-reducing sugar such as sucrose, lactose or trehalose. The amount of lyoprotectant generally included is such that, upon reconstitution, the resulting composition will be isotonic, although hypertonic or slightly hypotonic compositions also may be suitable. In addition, the amount of lyoprotectant should be sufficient to prevent an unacceptable amount of degradation and/or aggregation of the composition components upon lyophilization. A parenteral composition typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives are also present in some embodiments, such as, for example, anti-microbials, anti-oxidants, chelating agents, inert gases and the like.

A composition described herein, in some implementations, is formulated for controlled or sustained delivery in a manner that provides local concentration of the product (e.g., bolus, depot effect) and/or increased stability or half-life in a particular regional environment. The compositions may comprise a beta-2 adrenergic receptor agonist disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained-or controlled-delivery means are known and a variety of polymers have been developed and used for the controlled release and delivery of drugs. Sustained-release compositions also may include liposomes, which may be prepared by any of several methods known in the art. The carrier itself, or its degradation products, should be nontoxic in the target tissue and should not further aggravate the condition. This may be determined by routine screening in animal models of the target disorder or, if such models are unavailable, in normal animals. The beta-2 adrenergic receptor agonists disclosed herein may be microencapsulated.

Some embodiments of the composition comprise one or more sustained or controlled release agents known in the art for providing a sustained or controlled release of a beta-2 agonist and/or glucocorticosteroid, which are, for example, encapsulated in, bound to, and/or conjugated to the sustained or controlled release agent or carrier. In some embodiments, biocompatible, biodegradable sustained or controlled release compositions provide local tissue activity for weeks to months. Suitable sustained or controlled release agents or carriers are known in the art, for example, polymers, macromolecules, active ingredient conjugates, hydrogels, contaminations thereof, and the like. Some embodiments of the sustained release carrier target fat, for example, liposomes. Preferably, the sustained release materials are selected to facilitate delivery of a substantially equal amount of the active substance per unit time, particularly over the course of at least about 3 days, at least about 4 days, at least about 5 days, at least about 6 days, at least about 1 week, at least about 2 weeks, at least about 1 month, and up to one year or greater. Several rounds of injections of the sustained release composition can be made over time to treat a single area of a region.

In some embodiments, the sustained release agent comprises a polymer, for example, polylactides, polyglycolides, poly(lactide glycolides) polylactic acids, polyglycolic acids, polyanhydrides, polyorthoesters, polyetheresters, polycaprolactones, polyesteramides, polycarbonates, polycyanoacrylates, polyurethanes, polyacrylates, and blends, mixtures, or copolymers of the above, which are used to encapsulate, binds, or conjugate with the active ingredients(s) (e.g., beta agonists and/or glucocorticosteroids). Some embodiments of sustained release polymers comprise polyethylene glycol groups to which one or more of the active ingredients is conjugated. In some embodiments, the sustained release agent comprises poly(lactide glycolide) (PLGA, polylactic-co-glycolic acid)) copolymer 15. Some embodiments of the sustained release agent comprise one or more hydrogels known in the art, for example, modified alginates. Some embodiments of the sustained release agent comprise an albumin-based nano-particle carrier or excipient.

In some embodiments, the controlled release materials here release characteristics designed for the particular application of tissue reduction. In some embodiments, the sustained release or controlled release agent is formed into microparticles, such as microspheres, which are formulated as an injectable solution and/or gel. In some embodiments, the microparticles are from about 10 μm to about 100 μm in diameter and generally uniform in size. For example, in some embodiments, compositions comprising alginates and/or poly(lactide-co-glycolide)s 15 are provided as an injectable gel or processed into microspheres using methods known in the art. Other examples of suitable injectable biodegradable, biocompatible materials suitable for microparticle formation include chitosan, dextran, hydroxyapetite, and silicon.

Microspheres and/or microparticles are formed using any method known in the art, for example, by a solvent evaporation and/or emulsion polymerization. In some embodiments, the microspheres have average diameters of from about 5 μm to about 60 μm. In some embodiments, PLGA is manufactured with varying ratios of lactide to glycolide depending on the desired rate of release of the active ingredient(s). Because the rate of degradation of this copolymer is proportional to its crystallinity and the proportion of glycolide in the composition, non-racemic mixtures of the lactide and/or glycolide increase crystallinity and slow the rate of degradation. Higher proportions of glycolide increase the rate of degradation. In some embodiments, a ratio of about 65%-75% lactide to about 25%-35% glycolide provides active ingredients released over from about 2 weeks to about 45 days. In other embodiments, the ratio of lactide to glycolide is from about 0:100 to about 100:0, thereby providing other release rates.

Some embodiments of the microspheres or microparticles comprise hollow and/or porous interiors. In some embodiments, the microspheres comprise a solid or porous outer shell. Some embodiments of compositions comprising a porous outer shell and/or micro sphere exhibits a biphasic release profile of the active ingredient(s) with an initial release burst of the active ingredient(s), followed by a sustained release associated with degradation of the polymeric microspheres, wherein the active ingredient is a beta-2 adrenergic receptor agonist. The initial burst loads the tissue with an effective lipolytic/adipogenesis inhibitory concentration of the active ingredient(s), with the subsequent slower release maintaining the desired concentration. In some embodiments, the different microsphere structures and active ingredient release profiles optimize the treatment effect of adipose tissue and adipocytes through adrenergic receptor modulation. In some embodiments, sustained local tissue concentrations of long-acting selective beta-2 adrenergic agents, such as salmeterol are at concentrations from about 10 pM to about 10 μM.

In some embodiments, one or more of the composition ingredients, e.g., beta-2 adrenergic receptor agonist, are encapsulated, bound, and/or conjugated to the polymer at a ratio of about 5 to about 20% by mass compared to the polymer microspheres. The amount of ingredient as a mass percentage of the carrier (e.g., microparticles or microspheres), in many embodiments, is referred to as active ingredient loading. In many instances, loaded and loading refer to an ingredient substantially encapsulated, bound, and/or conjugated to a carrier. In some embodiments, the ingredient loading is up to about 75%. Thus, some compositions comprising one or more beta-2 adrenergically active ingredients, such as salmeterol, formoterol, and/or their physiologically acceptable salts and solvates, loaded on polymer microspheres at about 1 mg to about 20 mg of active ingredient per about 10 to about 200 milligrams of polymer. In some embodiments, a composition with this ingredient loading is sufficient for providing from about 15 days to about 45 days of active ingredient release at a concentration suitable to produce lipolysis and/or adipogenesis inhibition.

In some embodiments, two or more ingredients of a composition, including a beta-2 adrenergic receptor agonist, are loaded into the same microsphere, for example, in a liposome.

Thus, in some embodiments, a polymer encapsulating a glucocorticosteroid and beta-2 adrenergic receptor agonist is delivered simultaneously to the adipose tissue. Alternatively, two or more ingredients of a composition may be loaded on separate microspheres. The two types of microspheres are then mixed to obtain a composition with the desired ratio of beta-2 receptor agonist and glucocorticosteroid, then administered simultaneously. Alternatively, the two types of microspheres are administered sequentially.

In some embodiments, microspheres comprising one or more components or ingredients of the composition are administered in a physiologically acceptable liquid carrier. In some embodiments using needleless injection, the microparticulate compositions are injected as suspensions or as the powdered loaded microparticles, that is, without a liquid carrier.

In some embodiments, the composition is a controlled release composition comprising a controlled release carrier, a beta-2 adrenergic receptor agonist (e.g., long-acting), and/or a glucocorticosteroid. In some instances, the controlled release carrier reduces fat in the treatment of regional fat accumulation. In some embodiments, the beta-2 agonist is salmeterol and/or salmeterol xinafoate. In some embodiments, the controlled release carrier is a biodegradable polymer. In some embodiments, the biodegradable polymer comprises lactide and glycolide. In some embodiments, the biodegradable polymer is formulated as a microparticle. In some embodiments, the glucocorticoid is fluticasone, budesonide, and/or dexamethasone. In some embodiments, at least a portion of the composition is delivered to the treatment region through a needleless injection device. In additional embodiments, the needleless injection device promotes the lateral spread of the composition in the fat accumulation of the region. In many embodiments, the composition is delivered to the treatment region by subcutaneous injection.

Suitable and/or preferred composition formulations may be determined in view of the present disclosure and general knowledge of composition technology, depending upon the intended route of administration, delivery format, and desired dosage. Regardless of the manner of administration, an effective dose may be calculated according to patient body weight, body surface area, or treatment region size. Further refinement of the calculations for determining the appropriate dosage for treatment involving the compositions described herein are routinely made in the art and is within the ambit of tasks routinely performed in the art. Appropriate dosages may be ascertained through use of appropriate dose-response data.

### Administration

In one aspect, provided herein are compositions comprising beta-2 adrenergic receptor agonists for administration to a patient suitable for body contouring treatment, wherein the body contouring treatment functions to reduce regional fat deposits and/or adipose tissue in the region the composition is administered to. In some embodiments, the region is a central abdominal region. In some embodiments, the region is a submental region. In some embodiments, the beta-2 adrenergic receptor agonist is long-acting. In some embodiments, the beta-2 adrenergic receptor agonist is lipophilic.

In some embodiments, the composition is administered to a region having subcutaneous fat in a dosage comprising from about 1 nanogram to about 50 micrograms beta 2-adrenergic receptor agonist. In some instances, each dosage comprises from about 1 nanogram (ng) to about 50 microgram (μg), from about 1 ng to about 40 μg, from about 1 ng to about 30 μg, from about 1 ng to about 20 μg, from about 1 ng to about 10 μg, or from about 1 ng to about 5 μg beta-2 adrenergic receptor agonist. In some instances, each dosage comprises from about 1 ng to about 1 μg, from about 1 ng to about 100 ng, from about 1 ng to about 90 ng, from about 1 ng to about 80 ng, from about 1 ng to about 70 ng, from about 1 ng to about 60 ng, from about 1 ng to about 50 ng, from about 1 ng to about 40 ng, from about 1 ng to about 30 ng, from about 1 ng to about 25 ng, from about 1 ng to about 20 ng, from about 1 ng to about 15 ng, or from about 1 ng to about 10 ng beta-2 adrenergic receptor agonist. In some embodiments, each dosage is administered from about 1 to about 100 times to the region having subcutaneous fat during a session. In some embodiments, a total dosage administered during a session comprises from about 0.05 micrograms to about 50 micrograms beta-2 adrenergic receptor agonist. In some embodiments, a total dosage administered during a session comprises about 0.4 micrograms beta-2 adrenergic receptor agonist. In some instances, a total dosage administered during a session comprises from about 1 ng to about 50 μg, from about 1 ng to about 40 μg, from about 1 ng to about 30 μg, from about 1 ng to about 20 μg, from about 1 ng to about 10 μg, or from about 1 ng to about 5 μg beta-2 adrenergic receptor agonist. In some instances, a total dosage administered during a session comprises from about 1 ng to about 1,500 ng, from about 1 ng to about 1,400 ng, from about 1 ng to about 1,300 ng, from about 1 ng to about 1,200 ng, from about 1 ng to about 1,100 ng, from about 1 ng to about 1,000 ng, from about 1 ng to about 900 ng, from about 1 ng to about 800 ng, from about 1 ng to about 700 ng, from about 1 ng to about 600 ng, from about 1 ng to about 500 ng, from about 1 ng to about 400 ng, from about 1 ng to about 300 ng, from about 1 ng to about 200 ng, or from about 1 ng to about 100 ng, beta-2 adrenergic receptor agonist. In some embodiments, the beta-2 adrenergic receptor agonist comprises salmeterol, a salt or solvate of salmeterol, or a combination thereof. In some embodiments, the beta-2 adrenergic receptor agonist is salmeterol xinafoate.

In some embodiments, a course of treatment comprises one session per week, for a period of about 8 weeks. In some embodiments, each dosage is administered from about 1 cm to about 10 cm apart to the region having subcutaneous fat. In some embodiments, each dosage is administered about 4 cm apart. In some embodiments, the composition is administered to the abdomen. In some embodiments, the composition is administered between axial planes located at about +100 mm and about −150 mm, relative to the umbilicus. In some embodiments, the composition is administered between axial planes located at about +70 mm and about −90 mm, relative to the umbilicus. In some embodiments, the composition is administered between axial planes located at about +35 mm and about −70 mm, relative to the umbilicus.

A body contouring treatment for localized fat reduction, in various embodiments, includes weekly administration sessions of the composition over a period of about eight weeks by a clinician (e.g., physician). In some embodiments, the treatment regimen includes 1, 2, 3, 4, 5, 6, 7, 8, 9, 10, 11, 12, 13, 14, 15, 16, 17, 18, 19, 20, 21, 22, 23, 24 or more sessions. In some embodiments, each session occurs once per week. In some embodiments, 2, 3, 4, 5, 6, or 7 sessions occur per week. A session includes one or a plurality of injections performed during one clinician visit. In some embodiments, a treatment period is one treatment session. In other embodiments, a treatment period lasts 1, 2, 3, 4, 5, 6, 7 or 8 sessions. In additional or other embodiments, a treatment period is from about 1 to about 8 sessions, from about 2 to about 8 sessions, from about 3 to about 8 sessions, from about 4 to about 8 sessions, from about 5 to about 8 sessions, from about 6 to about 8 sessions, from about 1 to about 24 sessions, from about 2 to about 24 sessions, from about 3 to about 24 sessions, from about 4 to about 24 sessions, from about 5 to about 24 sessions, from about 6 to about 24 sessions, from about 7 to about 24 sessions, from about 8 to about 24 sessions, or any number within these ranges. A treatment regimen includes one or a plurality of treatment periods.

In some embodiments, each session comprises administering a dose of the composition to one or more areas of a treatment region. For example, in one session, from about 1 to about 100 doses of the composition are administered to different treatment areas of the treatment region. In some instances, each session comprises from about 1 to about 90, from about 1 to about 80, from about 1 to about 70, from about 1 to about 60, from about 1 to about 50, from about 1 to about 40, from about 1 to about 30 or from about 1 to about 20 individual doses to different treatment areas of the treatment region. In some embodiments, the administration includes using a small, 30-gauge needle in the target region, such as, the central abdomen or submental region. In some embodiments, the composition is administered using a 25, 26, 27, 28, 29, 30, 31, 32, 33, 34, or 35-gauge needle. Administration may be completed in approximately five minutes or less. In some implementations, a single treatment session comprising the administration of one or more doses of the composition is performed in less than about 20 minutes, less than about 15 minutes, less than about 10 minutes, less than about 5 minutes, from about 3 to about 5 minutes, from about 2 to about 5 minutes, from about 1 to about 5 minutes, or from about 1 to about 10 minutes.

Administration of a composition provided herein is performed by a clinician, which includes, without limitation, a dermatologist, plastic surgeon, primary care physician, OB/GYN, members of other specialties, some of whom perform liposuction, non-invasive fat reduction and other procedures for fat reduction, registered nurse, physician assistant, esthetician, optometrist, ophthalmologist, and any physician with a medical degree such as a M.D. or D.O.

A composition comprising a beta-2 adrenergic receptor agonist disclosed herein can be administered to a subject by any suitable administration route, including but not limited to, parenteral (intravenous, subcutaneous, intraperitoneal, intramuscular, intravascular, intrathecal, intravitreal, infusion, or local) and topical administration.

A composition comprising a beta-2 adrenergic receptor agonist disclosed herein is suitable for intramuscular, subcutaneous, peritumoral, or intravenous injection and can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, 0.9% saline, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Compositions suitable for subcutaneous injection also contain optional additives such as preserving, wetting, emulsifying, and dispensing agents. For intravenous injections, an active agent can be optionally formulated in aqueous solutions, preferably in physiologically compatible buffers such as Hank's solution, Ringer's solution, or physiological saline buffer. Parenteral injections optionally involve bolus injection or continuous infusion. Compositions for injection are optionally presented in unit dosage form, e.g., in ampoules or in multi-dose containers, with or without an added preservative. The composition described herein can be in a form suitable for parenteral injection as a sterile suspension, solution or emulsion in oily or aqueous vehicles, and contain formulatory agents such as suspending, stabilizing and/or dispersing agents. Compositions for parenteral administration include aqueous solutions of beta-2 adrenergic receptor agonists in water soluble form. Additionally, suspensions are optionally prepared as appropriate oily injection suspensions.

Alternatively or additionally, in some instances, the composition is administered to a treatment region via implantation of a membrane, sponge, or other appropriate material on to which beta-2 adrenergic receptor agonist disclosed herein has been absorbed or encapsulated. Where an implantation device is used, the device may be implanted into any suitable tissue, and delivery of beta-2 adrenergic receptor agonist disclosed herein may be directly through the device via bolus, or via continuous administration, or via catheter using continuous infusion.

The size of the treatment region will vary depending on the treatment region selected and the patient. In some embodiments, the treatment region has an area of approximately 1,000 cm², about 900 cm², about 800 cm², about 700 cm², about 600 cm², about 500 cm², about 400 cm², about 300 cm ², about 200 cm ², about 100 cm², about 90 cm², about 80 cm², about 70 cm², about 60 cm ², about 50 cm², about 40 cm², about 30 cm², about 20 cm² or about 10 cm². In some embodiments, the treatment area is less than about 1,000 cm², less than about 900 cm², less than about 800 cm², less than about 700 cm², less than about 600 cm², less than about 500 cm², less than about 400 cm², less than about 300 cm², less than about 200 cm², less than about 100 cm², less than about 90 cm², less than about 80 cm², less than about 70 cm², less than about 60 cm², less than about 50 cm², less than about 40 cm², or less than about 30 cm². In some embodiments, the treatment area is from about 10 cm² to about 1,000 cm², from about 10 cm² to about 900 cm², from about 10 cm ² to about 800 cm², from about 10 cm² to about 700 cm², from about 10 cm² to about 600 cm², from about 10 cm² to about 500 cm², from about 10 cm² to about 400 cm², from about 10 cm ² to about 300 cm², from about 10 cm² to about 200 cm², from about 10 cm² to about 100 cm², from about 1 cm² to about 1,000 cm², from about 1 cm² to about 900 cm², from about 1 cm² to about 800 cm², from about 1 cm² to about 700 cm², from about 1 cm² to about 600 cm², from about 1 cm² to about 500 cm², from about 1 cm² to about 400 cm², from about 1 cm² to about 300 cm², from about 1 cm² to about 200 cm², from about 1 cm² to about 100 cm², from about 1 cm² to about 90 cm², from about 1 cm² to about 80 cm², from about 1 cm² to about 70 cm², from about 1 cm² to about 60 cm², from about 1 cm² to about 50 cm², from about 1 cm² to about 40 cm ², from about 1 cm² to about 30 cm², from about 1 cm² to about 20 cm², or any integer within the provided ranges.

In instances wherein the treatment region is the central abdominal region, the treatment area, in many embodiments, is defined between axial planes at 100 mm above the umbilicus and at 150 mm below the umbilicus. The central abdominal treatment area, in some embodiments, is defined between axial planes at 90 mm above the umbilicus and at 140 mm below the umbilicus, axial planes at 90 mm above the umbilicus and at 130 mm below the umbilicus, axial planes at 90 mm above the umbilicus and at 120 mm below the umbilicus, axial planes at 80 mm above the umbilicus and at 120 mm below the umbilicus, axial planes at 80 mm above the umbilicus and at 110 mm below the umbilicus, axial planes at 80 mm above the umbilicus and at 100 mm below the umbilicus, axial planes at 70 mm above the umbilicus and at 100 mm below the umbilicus, axial planes at 70 mm above the umbilicus and at 90 mm below the umbilicus, axial planes at 60 mm above the umbilicus and at 90 mm below the umbilicus, axial planes at 50 mm above the umbilicus and at 80 mm below the umbilicus, axial planes at 40 mm above the umbilicus and at 80 mm below the umbilicus, axial planes at 40 mm above the umbilicus and at 70 mm below the umbilicus, axial planes at 35 mm above the umbilicus and at 70 mm below the umbilicus, or any number between the provided ranges.

In some embodiments, the composition is administered once per week (in one session) during the course of a treatment period. In some embodiments, the composition is administered twice per week in two sessions. In additional or other embodiments, the composition is administered 1, 2, 3, 4, 5, 6 or 7 times in one week. A session includes one or a plurality of injections performed during one clinician visit.

In some embodiments, a treatment period is one treatment session. In other embodiments, a treatment period lasts 1, 2, 3, 4, 5, 6, 7 or 8 sessions. In additional or other embodiments, a treatment period is from about 1 to about 8 sessions, from about 2 to about 8 sessions, from about 3 to about 8 sessions, from about 4 to about 8 sessions, from about 5 to about 8 sessions, from about 6 to about 8 sessions, from about 1 to about 24 sessions, from about 2 to about 24 sessions, from about 3 to about 24 sessions, from about 4 to about 24 sessions, from about 5 to about 24 sessions, from about 6 to about 24 sessions, from about 7 to about 24 sessions, from about 8 to about 24 sessions, or any number within these ranges. A treatment regimen includes one or a plurality of treatment periods.

In various embodiments, the composition comprising a beta-2 adrenergic receptor agonist is administered with a specified number and defined placement of subcutaneous injections in a region, such as a central abdominal or submental region.

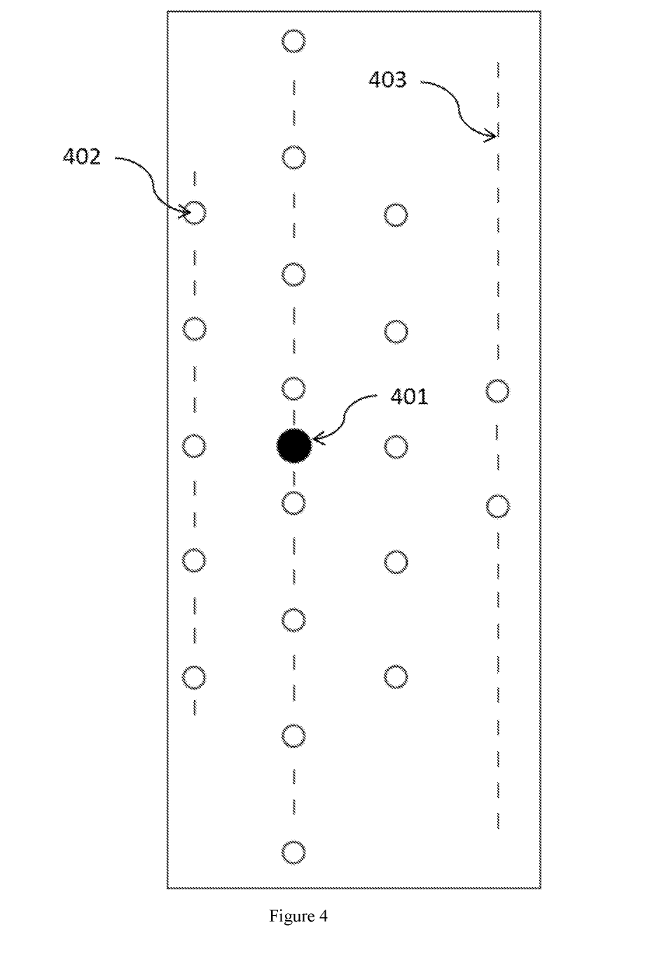

In various embodiments, the composition is administered using a stencil or tattoo. The stencil may be placed over the region and used as a template for administration, wherein holes of the stencil indicate administration areas or sites. The stencil is useful for defining treatment areas of the region. A temporary tattoo is useful for defining the location of the administration areas of the treatment region. In many instances, the temporary tattoo is water-based. The tattoo may be placed on the same treatment region for each treatment session to guide administration of the composition to the same areas each session. The tattoo or stencil may include any number of reference points, so that the tattoo or stencil is placed on substantially the same region of the body each session. For example, provided that the region is a central abdominal or submental region, a reference point on the tattoo or stencil may indicate the location of the umbilicus or middle of the chin, respectively. The stencil or tattoo functions to indicate one or more positions of putative administration areas of the treatment region. The administration areas may be spaced equidistance apart. In some embodiments, the distance between two adjacent administration areas is between 0.2 cm and 10 cm. In some embodiments, the distance between two adjacent administration areas is from about 0.5 cm to about 9 cm apart, from about 0.5 cm to about 8 cm apart, from about 0.5 cm to about 8 cm apart, from about 0.5 cm to about 7 cm apart, from about 0.5 cm to about 6 cm apart, from about 0.5 cm to about 5 cm apart or from about 0.5 cm to about 4 cm apart, as measured from the center of each administration area. The stencil or tattoo, in some embodiments, comprises from about 1 to about 100 administration sites, from about 1 to about 90 sites, from about 1 to about 80 sites, from about 1 to about 70 sites, from about 1 to about 60 sites, from about 1 to about 50 sites, from about 1 to about 40 sites, from about 1 to about 30 sites, from about 1 to about 20 sites, from 5 to about 100 sites, from about 5 to about 90 sites, from about 5 to about 80 sites, from about 5 to about 70 sites, from about 5 to about 60 sites, from about 5 to about 50 sites, from about 5 to about 40 sites, from about 5 to about 30 sites, from about 5 to about 20 sites, from about 10 to about 100 sites, from about 10 to about 90 sites, from about 10 to about 80 sites, from about 10 to about 70 sites, from about 10 to about 60 sites, from about 10 to about 50 sites, from about 10 to about 40 sites, from about 10 to about 30 sites or from about 10 to about 20 sites. The administration sites may be positioned together to form any shape of a total treatment region, such as a rectangle, square, triangle, circle or oval. The administration sites may be arranged in offset rows to form any pattern. The tattoo or stencil is configured so that all or a portion of the tattoo or stencil are utilized. As an example, the template for the tattoo is cut into smaller pieces, prior to application to a region of the body. As another example, the stencil is placed over the treatment region, and only a portion of the administration sites are used to guide administration of the composition. As another example, a stencil is provided with administration sites spaced 0.5 cm apart. In this example, the clinician may administer the composition using every other template site, so that the composition is administered every 1 cm. As another example, a stencil is provided with administration sites spaced 2 cm apart. Exemplary tattoos are shown in FIGS. 4 to 7.

In one embodiment, a stencil or tattoo is illustrated by FIG. 4. The reference point, indicated by the black dot, 401, is placed at the same area of the treatment region for each treatment-The clear circles are indicative of the positions of administration sites. In some embodiments, the administration is by subcutaneous injection to an area within each circle. In FIG. 4, there are 20 positions, 402, wherein a treatment is configured to be administered. In some instances, the composition is administered in 1, 2, 3, 4, 5, 6, 7, 8, 9, 10, 11, 12, 13, 14, 15, 16, 17, 18, 19 or 20 positions. The dashed lines of the stencil or tattoo, 403, in some instances, are indications of measurement. For example, each dash is indicative of a certain distance, such as cm.

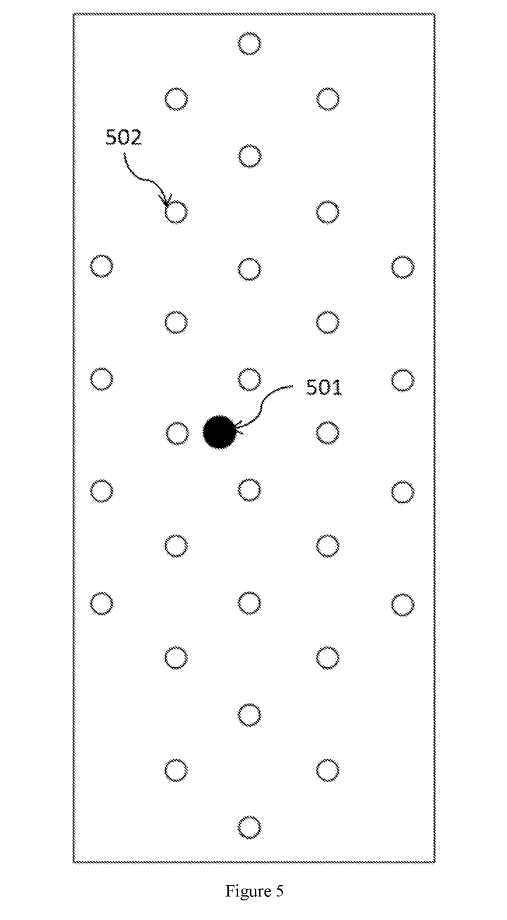

In one embodiment, a stencil or tattoos is illustrated by FIG. 5. The reference point, indicated by the black dot, 501, is placed at the same area of the treatment region for each treatment, and the clear circles, 502, are indicative of the positions of administration sites. In some embodiments, the administration is by subcutaneous injection to an area within each circle. In FIG. 5, there are 30 positions wherein a treatment is configured to be administered. In this figure, the administration sites together form an oval administration region. In some instances, the composition is administered in 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 positions.

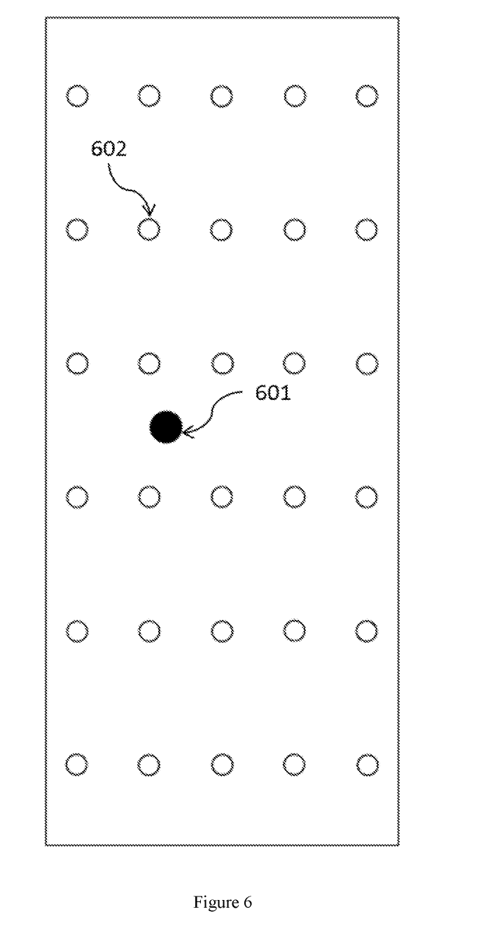

In one embodiment, a stencil or tattoos is illustrated by FIG. 6. The reference point, indicated by the black dot, 601, is placed at the same area of the treatment region for each treatment. The clear circles, 602, are indicative of the positions of administration sites. In some embodiments, the administration is by subcutaneous injection to an area within each circle. In FIG. 6, there are 30 positions wherein a treatment is configured to be administered. In this figure, the administration sites together form a rectangular administration region. In some instances, the composition is administered in 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 positions.

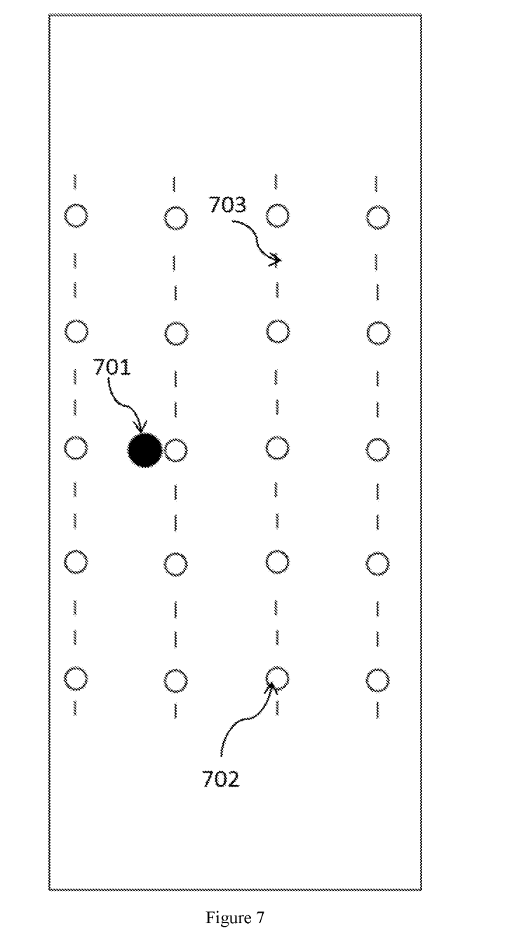

In one embodiment, a stencil or tattoos is illustrated by FIG. 7. The reference point, indicated by the black dot, 701, is placed at the same area of the treatment region for each treatment. The clear circles, 702, are indicative of the positions of administration sites. In some embodiments, the administration is by subcutaneous injection to an area within each circle. In FIG. 7, there are 20 positions wherein a treatment is configured to be administered. In some instances, the composition is administered in 1, 2, 3, 4, 5, 6, 7, 8, 9, 10, 11, 12, 13, 14, 15, 16, 17, 18, 19 or 20 positions. The dashed lines of the stencil or tattoo, 703, in some instances, are indications of measurement. For example, each dash is indicative of a certain distance, such as cm.

The tattoo or stencil, in many implementations, is helpful for obtaining measurements of the region prior to, during and/or after treatment.

In some embodiments, a composition comprising a beta-2 adrenergic receptor agonist is administered in combination with additional cosmetic and/or therapeutic treatments. For example, additional treatments include, without limitation, exercise, diet, surgical options (e.g., lipoplasty, liposuction), non-surgical options (e.g., energy-based medical devices), and any method designed to remove, damage, reduce or kill fat cells. In some embodiments, a treatment regimen includes recommendations that a patient receiving the composition follow a diet and/or exercise program. In some instances, the combination of eating well and the administration of the composition has a greater effect on reducing subcutaneous fat than eating well alone or the composition alone. In some embodiments, the effect is a 1% to 50%, 1% to 40%, 1% to 30%, 1% to 20%, 1% to 10% or a 1% to 5% decrease in subcutaneous fat volume in the treatment region. In some instances, the combination of exercise and the administration of the composition has a greater effect on reducing subcutaneous fat than exercise alone or the composition alone. In some embodiments, the effect is a 1% to 50%, 1% to 40%, 1% to 30%, 1% to 20%, 1% to 10% or a 1% to 5% decrease in subcutaneous fat volume in the treatment region. In some instances, the combination of eating well, exercise and the administration of the composition has a greater effect on reducing subcutaneous fat than eating well alone, exercising alone or the composition alone. In some embodiments, the effect is a 1% to 50%, 1% to 40%, 1% to 30%, 1% to 20%, 1% to 10% or a 1% to 5% decrease in subcutaneous fat volume in the treatment region. In other instances, the patient does not alter their lifestyle during the course of treatment.

A body contouring treatment, in many instances, is performed during any time of day or week, with the patient able to return to regular daily activities immediately after administration. A body contouring treatment provided herein, in many instances, has reduced risks and complications as compared to cosmetic body contouring surgeries. Risks and complications for surgeries such as liposuction include infections, embolisms, puncture wounds in organs, serum pooling in treated area, nerve damage, swelling, skin death, toxicity from anesthesia and fatalities. Additional complications include uneven contours, rippling or loose skin, irregular pigmentation, unfavorable scarring, skin discoloration, bleeding or hematoma, deep vein thrombosis, cardiac and pulmonary complications, and possibility of corrective surgery. The body contouring treatments provided herein, in many instances, do not cause extensive pain or require recovery time, such as is experienced with surgical procedures. Following a cosmetic surgical procedure, such as liposuction, patients may expect pain and swelling for several weeks and even months, and patients may be required to wear compression garments for several weeks to control swelling and drainage. The body contouring treatments described herein, in many instances, do not provide the potential for undesirable results such as surgical cosmetic treatments. Undesirable results from surgical cosmetic procedures include, without limitation, skin dimpling (the skin takes on the appearance of cellulite), and weight gain after surgery which may be stored in regions of the body which were not treated in greater concentrations, providing undesirable total body contour. An additional improvement over surgical procedures, includes, in various instances, no formation of scar tissue in the treated area, allowing for multiple treatments if desirable. Side effects from non-cosmetic surgeries such as cryolipolysis and high frequency ultrasound include, in some instances, umbilical hernia, nerve damage, extended and debilitating pain and burns.

### Treatment Efficacy

In some embodiments, a statistically significant reduction in bulging due to subcutaneous fat, or a reduction in the appearance of fat, is measureable as soon as four weeks from initial treatment (e.g., the first treatment session). Treatment benefits may persist for a minimum of three months post-treatment. Treatment benefits are measured by a clinician, a patient, or both a clinician and the patient. In some embodiments, treatment benefits are cosmetic, improving a patient's appearance.

Accumulation of fat stores in humans can occur unevenly in the body, which to certain individuals is considered to be a cosmetic blemish. A cosmetic treatment, in many embodiments, refers to a composition comprising a beta-2 agonist provided herein which will improve the cosmetic appearance at the regional site of treatment, for example, a central abdominal or submental region. It is to be understood that cosmetic appearance can vary from subject to subject, due to numerous factors including, for example, age, weight, general condition of the subject, the condition being treated, the severity of the condition being treated, and the judgment of the patient and/or clinician. A body contouring treatment, in many instances, is carried out to achieve a cosmetically desirable body shape. Exemplary measurements of cosmetic efficacy include cosmetic waist or abdomen reduction or improvement in region contour (e.g., reduction of “turkey neck” for patients treated in the submental region). This reduction, in some instances, is at least about 0.5 cm in the treatment region. This reduction, in some instances, is at least about a 1% decrease in subcutaneous fat volume in the treatment region.

In some embodiments, treatment benefits are therapeutic. A therapeutic effect, for example, is a reduction in adipose tissue and/or reduction of regional fat deposits in the treatment region. Efficacy of treatments using the compositions provided herein does not necessarily require a reduction in weight of the patient or alteration in exercise routine by the patient, but rather the efficacy of the compositions and methods of treatment described herein is, in many circumstances, independent of these factors and influences. Accordingly, provided herein, in certain embodiments, are compositions in optimal dosage amounts that provide therapeutic effect in a patient. The therapeutic effect for reducing regional fat deposits and/or adipose tissue, in some instances, includes a reduced risk of producing cardiovascular side effects. In some embodiments, administration of the composition to a lipoma reduces the size of the lipoma. Compositions provided herein comprising beta-2 agonists for subcutaneous administration, in some embodiments, provide a therapeutic effect to a regional fat deposit and a reduced or minimized risk of producing the side effects associated with the use of other long-acting beta-2 agonists or long-acting beta-2 agonists administered by other methods. Such side effects include paradoxical bronchospasm, high blood pressure, abnormal heart rhythm, abnormally low blood pressure, an increase in asthma related conditions, bronchospasm, inflammation of the lining of the stomach and intestines, involuntary quivering, fast heartbeat, chest pain, and giant hives.

Treatment efficacy, in various embodiments, is assessed through a variety of physical and observational measures, including, without limitation, tape measurement, two-dimensional ultrasound, and clinical outcome assessments such as patient-reported outcome and clinician-reported outcome. In some cases, the tape measurement is guided using a laser.

In one aspect, provided herein are methods of determining the effect of a body contouring treatment in a region of a patient by performing one or more assessments to evaluate a reduction of subcutaneous fat in said region or a reduction in visual appearance of fat, provided that an assessment is conducted after administration of a composition comprising an effective amount of a beta-2 adrenergic receptor agonist to the region of the body comprising excess subcutaneous fat. In various embodiments, the patient is non-obese. The methods include patient self-assessment and clinician rating of body contour (e.g., excess subcutaneous fat, bulging), as well as physical measures of excess subcutaneous fat and a questionnaire that measures the impact of fat on patients.

An exemplary patient self-assessment comprises the patient evaluating the visual appearance of the region using a descriptive phrase. The descriptive phrases, in various instances, are converted to a numeric scale, for example, each phrase receives a score of 0, 1, 2, 3, or 4. In some instances, a region having a score with a low number (e.g., 0 or 1) has a lower amount of subcutaneous fat than a region having a score with a high number (e.g., 3 or 4). In some instances, a region having a score with a low number (e.g., 0 or 1) has a lower amount of visible fat (or protruding soft tissue comprising skin, fat, or skin and fat) than a region having a score with a high number (e.g., 3 or 4). In some embodiments, a patient self-assessment involves the patient rating the contour and/or appearance of the region using a numeric scale. In some embodiments, a patient self-assessment involves the patient comparing their region to a set of regional photos in a photonumeric scale. In one aspect, treatment efficacy is indicated in patients who show at least a 1-point/grade improvement (e.g., decrease) on a patient self-assessment scale.

In some embodiments, a patient self-assessment comprises the patient evaluating the amount of bulging in the region using a descriptive phrase. As an example, the region is a central abdominal region. In some embodiments, the patient rates the amount of bulging in the region using a numeric scale. In some embodiments, the patient compares the amount of bulging in the region to a photonumeric scale. In an embodiment wherein the patient uses descriptive phrases, such phrases include, without limitation, flat, almost flat, slight bulge or not flat, bulge, or big bulge. The descriptive phrases, in various instances, are converted to a numeric scale, for example, flat receives a score of 0, almost flat receives a score of 1, slight bulge or not flat receives a score of 2, bulge receives a score of 3 and big bulge receives a score of 4. In some cases, the descriptive phrases are converted to a numeric scale by a clinician.

In one aspect, treatment efficacy is indicated in patients who show at least a 1-point/grade improvement in bulging, or achieve flattening on a patient self-assessment scale. As an example, a patient responsive to treatment who has described the treatment region prior to treatment as a big bulge, will describe the treatment region after treatment as a bulge, slight bulge or not flat, almost flat or flat. As another example, a patient responsive to treatment who has described the treatment region prior to treatment as a bulge, will describe the treatment region after treatment as slight bulge or not flat, almost flat or flat. As another example, a patient responsive to treatment who has described the treatment region prior to treatment as slight bulge or not flat, will describe the treatment region after treatment as almost flat or flat. As another example, a patient responsive to treatment who has described the treatment region prior to treatment as almost flat, will describe the treatment region after treatment as flat.

A patient self-assessment, in many circumstances, involves a patient answering a questionnaire on the contour of the treatment region. In some cases, the questionnaire evaluates the impact of bulging in the treatment region on the patient. The questions may be answered on an ordinal scale. In some embodiments, a patient questionnaire comprises a question regarding the importance of reducing subcutaneous fat or reducing the appearance of fat in the treatment region. In some embodiments, a patient questionnaire comprises a question regarding the importance of flattening of the treatment, for example, wherein the treatment region is a central abdominal region. In some embodiments, a patient questionnaire comprises a question on how self-conscious the patient is on how the treatment region looks. In some embodiments, a patient questionnaire comprises a question on how much bulging or fat the patient perceives in the treatment region. In some embodiments, a patient questionnaire comprises a question on how bothered the patient is about the bulging or contour in the treatment region. In some embodiments, a patient questionnaire comprises a question on whether the treatment region makes the patient look less attractive. In some embodiments, a patient questionnaire comprises a question on whether the patient wears certain clothes to hide or disguise how the treatment area looks. In some embodiments, a patient questionnaire comprises a question regarding if other people saw the treatment region, would the patient think the other people would judge the patient negatively. In some embodiments, a patient questionnaire comprises a question on whether due to the bulging in the treatment region, the patient feels self-conscious when wearing certain types of clothing. In some embodiments, a patient questionnaire comprises a question on whether the bulge in the treatment region limits the clothes the patient can buy or wear. In some embodiments, a patient questionnaire comprises a question regarding patient satisfaction with the flatness of the treatment region.

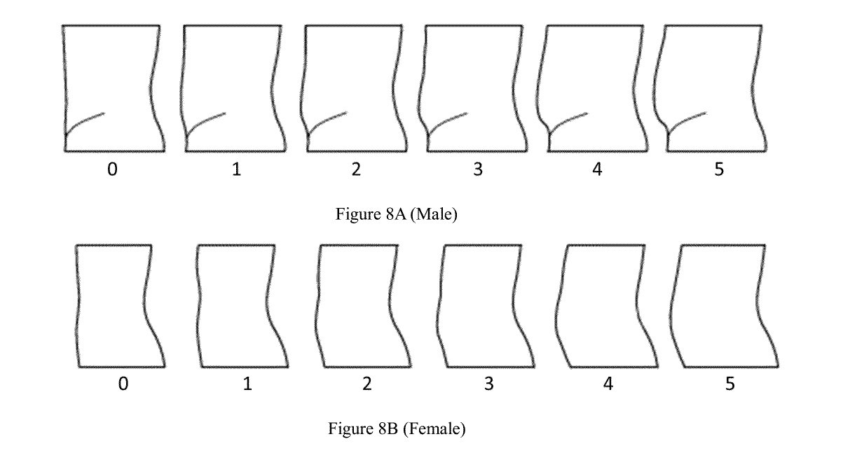

In one aspect, a patient assessment comprises the patient rating the amount of visible fat (e.g., bulging) and/or contour of the treatment region on a photonumeric scale, a Patient-reported Photonumeric Scale, or PPnS. Using the PPnS, the patient performs a match-to-sample task from one of two gender-specific scales of lateral profile pictures showing progressively larger regions of fat or bulge. In some embodiments, the region is a submental region. In some embodiments, the bulging region is in the central abdominal region. Example embodiments of two six-point photonumeric scales are shown in FIGS. 8A and 8B: each figure has lateral profile torso pictures with progressively larger abdominal bulges. FIG. 8A is illustrative of males having various ranges of central abdominal bulging and FIG. 8B is illustrative of females having various ranges of central abdominal bulging.

In one aspect, a clinician assessment of a patient's treatment region comprises the clinician evaluating the contour of the region using a descriptive phrase. The descriptive phrases, in various instances, are converted to a numeric scale, for example, a contour due to large amounts of excess subcutaneous fat will have a high number on the numeric scale as compared to a contour having little or no excess subcutaneous fat which will have a low number on the numeric scale. In some embodiments, a clinician assessment involves the clinician rating the contour of the region using a numeric scale. In one aspect, treatment efficacy is indicated in patients who show at least a 1-point/grade improvement in body contour on a clinician assessment scale. A clinician-reported scale, in various embodiments, involves a clinician rating the contour of the region using a photonumeric scale or CPnS, wherein the clinician performs a match-to-sample task from one of two gender-specific scales of lateral profile pictures with progressively larger amounts of visible fat. In many embodiments, the clinician-reported photonumeric scale and the patient-reported photonumeric scale use the same photographs. In some cases, the region described in the clinician assessment has an excess of subcutaneous fat and/or has visible bulging. For example, the region is the central abdominal region. For cases wherein the region is a central abdominal region, descriptive phrases include, without limitation, flat, almost flat, slight bulge or not flat, bulge, or pronounced bulge. These descriptive phrases, in various instances, are converted to a numeric scale, for example, flat receives a score of 0, almost flat receives a score of 1, slight bulge or not flat receives a score of 2, bulge receives a score of 3 and pronounced bulge receives a score of 4. An example of a six-point photonumeric scale is shown in FIGS. 8A and 8B, wherein FIG. 8A is illustrative of males with various ranges of central abdominal bulging and FIG. 8B is illustrative of females with various ranges of central abdominal bulging. In some embodiments, the CPnS and PPnS use the same photographs. For example, the photonumeric scales of FIGS. 8A and 8B are useful in both a CPnS and a PPnS.

In some embodiments, treatment efficacy is indicated in patients who show at least a 1-point/grade improvement in body contour, bulging, and/or appearance of fat on a clinician assessment scale (CPnS). In one aspect, clinically-meaningful responders to treatment are those patients who show at least a 2-point/grade improvement in body contour or the appearance of fat on a CPnS. In some embodiments, the CPnS is a 3, 4, 5, 6, 7, 8, 9 or 10-point photonumeric scale, wherein the numerical value correlates directly with the degree of bulging and/or fat in the treatment region. In one embodiment, the scale is a 6-point photonumeric scale. In many implementations, the photonumeric scale is different for a male and a female. In one example, a patient responsive to treatment who has a CPnS score of 5 on a scale of 0 to 5 prior to treatment, will have a CPnS score of 0, 1, 2, 3 or 4 after treatment. In another example, a patient responsive to treatment who has a CPnS score of 4 on a scale of 0 to 5 prior to treatment, will have a CPnS score of 0, 1, 2 or 3 after treatment. In another example, a patient responsive to treatment who has a CPnS score of 3 on a scale of 0 to 5 prior to treatment, will have a CPnS score of 0, 1 or 2 after treatment. In another example, a patient responsive to treatment who has a CPnS score of 2 on a scale of 0 to 5 prior to treatment, will have a CPnS score of 0 or 1 after treatment. In another example, a patient responsive to treatment who has a CPnS score of 1 on a scale of 0 to 5 prior to treatment, will have a CPnS score of 0 after treatment.

In some cases wherein the treatment region is the central abdominal region, a patient responsive to treatment who has had the region described prior to treatment as a pronounced bulge, will have the region after treatment described as a bulge, slight bulge or not flat, almost flat or flat. As another example, a patient responsive to treatment who has a region prior to treatment described as a bulge, will have the region after treatment described as slight bulge or not flat, almost flat or flat. As another example, a patient responsive to treatment who has had the region prior to treatment described as slight bulge or not flat, will have the region described as almost flat of flat after treatment. As another example, a patient responsive to treatment who has had the region prior to treatment described as almost flat, will have the region described as flat after treatment. In some embodiments, the descriptive phrases of the clinician assessment for describing the central abdominal region are converted to a numeric scale. For example, a score of 0 indicates a low level of bulging or flat and a score of 4 indicates a high level of bulging. In one example, the phrase pronounced bulge is scored a 4, bulge is scored a 3, slight bulge or not flat is scored a 2, almost flat is scored a 1 and flat is scored a 0.

In one aspect, an assessment of treatment is evaluated using a tape measure procedure. In some cases, the tape measurement is guided using a laser. In an embodiment wherein the treatment region is a central abdominal region, a laser-guided manual tape measure provides a precise and reproducible measure of circumference at different levels on the abdomen. This method is performed, in exemplary embodiments, using patient standardization instructions, such as positioning, posture and breathing. In some embodiments, the method utilizes a self-tensioning tape measure. In some embodiments, the method utilizes a treatment area grid, consisting of a temporary tattoo or stencil applied to the region, such as the central abdomen. In some embodiments, the method utilizes a tripod-mounted laser level to assure horizontal placement of the tape measure. In an embodiment wherein the treatment region is an arm region, a laser-guided manual tape measure provides a precise and reproducible measure of circumference at different levels on the arm. This method is performed, in exemplary embodiments, using patient standardization instructions, such as positioning and posture. In some embodiments, the method utilizes a self-tensioning tape measure. In some embodiments, the method utilizes a treatment area grid, consisting of a temporary tattoo or stencil applied to the region. In some embodiments, the method utilizes a tripod-mounted laser level to assure horizontal placement of the tape measure. The tattoo or stencil, in many implementations, is helpful for obtaining measurements of the region prior to, during and/or after treatment. Regarding the laser-guided tape measure procedure, the reproducibility of the procedure beyond what is obtained with a standard manual tape measure, in many circumstances, is enhanced with a tattoo placed on the treatment region. By utilizing the laser, tattoo, and tape measure, the measurements are made at the same or relatively the same position each session.

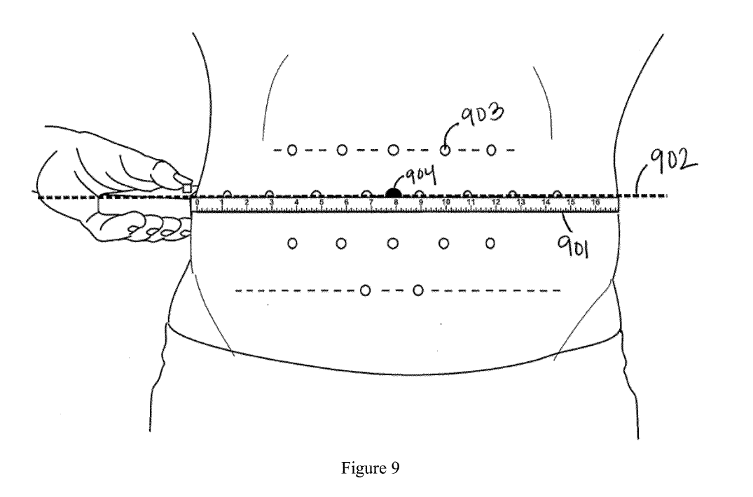

The tattoo or stencil, in many implementations, is helpful for obtaining measurements of the region prior to, during and/or after treatment. Regarding the laser-guided tape measure procedure, the reproducibility of the procedure beyond what is obtained with a standard manual tape measure, in many circumstances, is enhanced with a tattoo placed on the treatment region. An example of a laser-guided manual tape measure is shown in FIG. 9, which illustrates a measurement of central abdominal bulging circumference by laser-guided tape measure procedure using a tattoo. The figure shows a self-tensioning tape measure, 901. The figure shows a guidance beam, 902, emitted from a tripod-mounted laser level (not shown). As shown in the figure, the tattoo on the patient's abdomen comprises a plurality of open circles, 903, indicative of administration sites. The tattoo in this example comprises four rows of administration sites. The second row is lined up with the umbilicus. The dark reference dot, 904, of the tattoo indicates the reference point to the umbilicus. In this example, a laser guides the position of the tape measure. By utilizing the laser, tattoo, and tape measure, the abdominal circumference may be measured at the same or relatively the same position each session.

In one aspect, a patient suitable for body contouring treatment is evaluated for treatment efficacy using a skin-pinch caliper. In another aspect, a patient suitable for body contouring treatment is evaluated for treatment efficacy using a constant-tension tape measure (with or without laser-guiding). In one aspect, assessment of treatment is evaluated using three-dimensional digital photographic imaging to measure changes in subcutaneous fat circumference and volume. In some embodiments, three-dimensional digital sterophotogrammetry is an objective procedure to measure bulging and therefore treatment efficacy. In some embodiments, Magnetic Resonance Imaging, or MRI, is a procedure to measure bulging and subcutaneous fat parameters, such as abdominal circumference and subcutaneous fat volume, and is useful for assessing treatment efficacy. In some embodiments, three-dimensional digital sterophotogrammetry is an objective procedure to measure bulging and therefore treatment efficacy.

In one aspect, treatment efficacy is indicated in patients who experience a reduction in subcutaneous fat or the appearance of fat (in some instances, when compared to placebo) expressed as a mean change from baseline or as a percentage of reduction in fat. In some cases, treatment efficacy is indicated in patients who experience a reduction in bulging, for example, a reduction in central abdominal bulging. The reduction in fat, in many instances, is measured directly or indirectly using a tape measure. In some cases, the tape measurement is guided using a laser. In some embodiments, a significant reduction is at least about 0.2 cm, at least about 0.4 cm, at least about 0.6 cm, at least about 0.8 cm, at least about 1.0 cm, at least about 1.2 cm, at least about 1.4 cm, at least about 1.6 cm, at least about 1.8 cm, at least about 2 cm, at least about 3 cm, at least about 4 cm or at least about 5 cm. The significance of the reduction is, in many instances, dependent on the location of the treatment region. For example, a significant reduction in the submental region may be about 0.5 cm and a significant reduction in the arm region may be about 1 cm. For the central abdominal region, a reduction, in many instances, is a reduction in umbilical circumference. In some embodiments, provided that the treatment region is the central abdominal region, the central abdominal circumference reduction after treatment is at least about 0.5 cm, at least about 1 cm, at least about 1.5 cm, at least about 2 cm, at least about 2.5 cm, at least about 3 cm, at least about 3.5 cm, at least about 4 cm, at least about 4.5 cm or at least about 5 cm. In some embodiments, provided that the treatment region is the central abdominal region, the central abdominal circumference reduction after treatment is from about 0.5 cm to about 5 cm, from about 0.5 cm to about 4 cm, from about 0.5 cm to about 3 cm, from about 1 cm to about 5 cm, from about 1 cm to about 4 cm or from about 1 cm to about 3 cm. The decrease in circumference is dependent on the starting circumference of the abdominal region, wherein an individual with an almost flat abdominal bulge does not have the same starting circumference as an individual having a big bulge.

In another aspect, treatment efficacy is indicated in patients who experience a decrease in subcutaneous fat in the region of treatment. The decrease, in various embodiments, is measured as a percentage of decrease in subcutaneous fat. A tape measure, in many instances, is utilized to measure the subcutaneous fat prior to and after treatment. In some cases, the tape measurement is guided using a laser. The percentage decrease, in some embodiments, is at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 10%, at least about 11%, at least about 12%, at least about 13%, at least about 14%, at least about 15% or at least about 20%. The percentage of reduction is, in most instances, dependent on the location of the treatment region. In some embodiments, provided that the treatment region is the submental region, the percentage decrease is at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9% or at least about 10%. In some embodiments, provided that the treatment region is the submental region, the percentage decrease is from about 1% to about 10%, from about 1% to about 9%, from about 1% to about 8%, from about 1% to about 7%, from about 1% to about 6%, from about 1% to about 5%, from about 1% to about 4% or from about 1% to about 3%. The percentage decrease is dependent on the starting volume of the region.

For example, a significant reduction in the submental region may be about 0.5 cm and a significant reduction in the arm region may be about 1 cm. In another aspect, treatment efficacy is indicated in patients who experience a decrease in subcutaneous fat in the region of treatment. The decrease, in various embodiments, is measured as a percentage of decrease in subcutaneous fat or bulging. A tape measure, in many instances, is utilized to measure the bulging prior to and after treatment. In some cases, the tape measurement is guided using a laser. In some cases, the bulging is measured using a caliper. In some cases, the bulging is measured using magnetic resonance imaging. The percentage decrease, in some embodiments, is at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 10%, at least about 11%, at least about 12%, at least about 13%, at least about 14%, at least about 15% or at least about 20%. The percentage of reduction is, in most instances, dependent on the location of the treatment region. In some embodiments, provided that the treatment region is the submental region and the bulging is measured using a caliper or magnetic resonance imaging, the percentage decrease is at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9% or at least about 10%. In some embodiments, provided that the treatment region is the submental region, the percentage decrease is from about 1% to about 10%, from about 1% to about 9%, from about 1% to about 8%, from about 1% to about 7%, from about 1% to about 6%, from about 1% to about 5%, from about 1% to about 4% or from about 1% to about 3%. The percentage decrease is dependent on the starting volume of the region, wherein an individual with a low level of subcutaneous fat does not have the same starting volume as an individual having a big bulge.

In some embodiments, provided that the treatment region is the central abdominal region, the percentage decrease is at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9% or at least about 10%. In some embodiments, provided that the treatment region is the central abdominal region, the percentage decrease is from about 1% to about 10%, from about 1% to about 9%, from about 1% to about 8%, from about 1% to about 7%, from about 1% to about 6%, from about 1% to about 5%, from about 1% to about 4% or from about 1% to about 3%. The percentage decrease is dependent on the starting volume of the abdominal region, wherein an individual with an almost flat abdominal bulge does not have the same starting volume as an individual having a big bulge.

In another aspect, treatment efficacy is indicated in patients who experience a mean change from baseline in subcutaneous fat volume. In some embodiments, the change (decrease in fat volume) after treatment is from about 1 cubic centimeters (cc) to about 1,000 cc, from about 1 cc to about 950 cc, from about 1 cc to about 900 cc, from about 1 cc to about 850 cc, from about 1 cc to about 800 cc, from about 1 cc to about 750 cc, from about 1 cc to about 700 cc, from about 1 cc to about 650 cc, from about 1 cc to about 600 cc, from about 1 cc to about 550 cc, from about 1 cc to about 500 cc, from about 1 cc to about 450 cc, from about 1 cc to about 400 cc, from about 1 cc to about 350 cc, from about 1 cc to about 300 cc, from about 1 cc to about 250 cc, from about 10 cc to about 1,000 cc, from about 10 cc to about 950 cc, from about 10 cc to about 900 cc, from about 10 cc to about 850 cc, from about 10 cc to about 800 cc, from about 10 cc to about 750 cc, from about 10 cc to about 700 cc, from about 10 cc to about 650 cc, from about 10 cc to about 600 cc, from about 10 cc to about 550 cc, from about 10 cc to about 500 cc, from about 10 cc to about 450 cc, from about 10 cc to about 400 cc, from about 10 cc to about 350 cc, from about 10 cc to about 300 cc, from about 10 cc to about 250 cc, from about 100 cc to about 1,000 cc, from about 100 cc to about 950 cc, from about 100 cc to about 900 cc, from about 100 cc to about 850 cc, from about 100 cc to about 800 cc, from about 100 cc to about 750 cc, from about 100 cc to about 700 cc, from about 100 cc to about 650 cc, from about 100 cc to about 600 cc, from about 100 cc to about 550 cc, from about 100 cc to about 500 cc, from about 100 cc to about 450 cc, from about 100 cc to about 400 cc, from about 100 cc to about 350 cc, from about 100 cc to about 300 cc, from about 100 cc to about 250 cc, or any value within the provided ranges. The volume of reduction, in most instances, is dependent on the location of the treatment region. The volume of reduction, in some embodiments, is measured using a tape measure. In some cases, the tape measurement is guided using a laser. In some embodiments, provided that the treatment region is the submental region, the reduction in volume after treatment is at least about 1 cc, at least about 2 cc, at least about 3 cc, at least about 4 cc, at least about 5 cc, at least about 6 cc, at least about 7 cc, at least about 8 cc, at least about 9 cc, at least about 10 cc, at least about 15 cc, at least about 20, at least about 30 cc, at least about 40 cc, at least about 50 cc, at least about 60 cc, at least about 70 cc, at least about 80 cc, at least about 90 cc, at least about 100 cc, at least about 120 cc, at least about 140 cc, at least about 160 cc, at least about 180 cc, or at least about 200 cc. In some embodiments, provided that the treatment region is the central abdominal region, the reduction in abdominal volume after treatment is at least about 50 cc, at least about 100 cc, at least about 150 cc, at least about 200 cc, at least about 250 cc, at least about 300 cc, at least about 350 cc, at least about 400 cc, at least about 500 cc, at least about 600 cc, at least about 800 cc or at least about 1,000 cc. The decrease in volume is dependent on the starting volume of the abdominal region, wherein an individual with an almost flat abdominal bulge does not have the same starting volume as an individual having a big bulge. In one aspect, treatment efficacy is not dependent on mean weight change as measured prior to and after treatment. For example, a patient experiences a 1 cm decrease in fat, e.g., submental fat due to excess subcutaneous fat, and does not experience total body weight loss. In additional embodiments, a patient experiences a decrease in bulging (decrease in volume, percentage) without a total body weight loss.

In one aspect, a body contouring treatment comprising the administration of a composition comprising a beta-2 adrenergic receptor agonist results in a similar reduction (e.g., within 10%) in treatment volume as a body contouring treatment comprising limited-volume liposuction, provided that the patient populations and treatment areas are similar. A similar patient population is a patient population all having similar bulging ratings based on patient self-assessment, CPnS, tape measure, or any assessment provided herein. In some cases, the tape measurement is guided using a laser. Similar treatment areas include areas having significantly similar (e.g., within 10%) dimensions.

Treatment efficacy, in various embodiments, is measured at any point after the first treatment session and up until one year after treatment. In many embodiments, treatment efficacy is measured after four weeks of treatment completion and up until six months after treatment. In some embodiments, treatment efficacy is measured three months after the first treatment session. In some embodiments, treatment efficacy is measured 1, 2, 3, 4, 5, 6, 7, 8, 9, 10, 11, 12, 13, 14, 15, 16, 17, 18, 19, 20, 21, 22, 23 or 24 weeks after an initial treatment session. In some embodiments, treatment efficacy is measured 25, 26, 27, 28, 29, 30, 31, 32, 33, 34, 35, 36, 37, 38, 39, 40, 41, 42, 43, 44, 45, 46, 47, 48, 49, 50, 51, or 52 weeks after an initial treatment session. In some embodiments, treatment efficacy is measured 1, 2, 3, 4, 5, 6, 7, 8, 9, 10, 11, 12, 13, 14, 15, 16, 17, 18, 19, 20, 21, 22, 23 or 24 weeks after completion of treatment. In some embodiments, treatment efficacy is measured 25, 26, 27, 28, 29, 30, 31, 32, 33, 34, 35, 36, 37, 38, 39, 40, 41, 42, 43, 44, 45, 46, 47, 48, 49, 50, 51, or 52 weeks after completion of treatment.

### Kits

According to one aspect of the subject matter, provided is a kit comprising one or more materials useful for treatment of a body region that has bulging due to subcutaneous fat and/or a visual appearance of fat due to excess subcutaneous tissue. In some embodiments, the kit comprises a pharmaceutically acceptable formulation of a composition comprising a beta-2 adrenergic receptor agonist. Non-limiting examples of beta-2 adrenergic receptor agonists include salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, and salts, solvates, polymorphs, and combinations thereof. In some embodiments, the kit comprises a tattoo or stencil to guide the administration of a composition to the body region for treatment. In some embodiments, the kit includes assessment materials for determining patient suitability for treatment. In some embodiments, the kit includes assessment materials for determining treatment efficacy. In some embodiments, the kit provides instructions for administering a composition to the region comprising excess subcutaneous fat, instructions for using a tattoo or stencil, and/or instructions for performing one or more assessments disclosed herein.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLES

### Example 1

### Phase 2 Clinical Trial: RESET

A randomized, placebo-controlled, multi-center Phase 2 dose-ranging clinical trial.

Methods: A randomized, double-blind, placebo-controlled study was conducted to evaluate the safety and efficacy of a composition comprising the long-acting beta-2 receptor agonist salmeterol xinafoate. This study was reviewed and approved by a central IRB (Chesapeake IRB, Columbia, Md.) and written informed consent was received from all study subjects. The study enrolled 513 adult subjects with central abdominal bulging at 31 investigative sites across the U.S. Subjects were required to be non-obese (BMI<30 kg/m²), have a focal area of central abdominal bulging due to excess subcutaneous fat (˜400 cm² area around the umbilicus), have adequate skin elasticity, have at least a rating of “slight bulge, not flat” on both the 5-point Patient- and Clinician-Global Abdominal Perception Scales (P-GAPS and C-GAPS) at Screening and Day 1, and who were at least slightly dissatisfied with their abdominal bulging in the treatment area. The P-GAPS includes the ratings: flat, almost flat, slight bulge/not flat, bulge and bulge.

Subjects were randomized to receive 1 of 3 doses of salmeterol xinafoate (0.4, 1.0, or 4.0 μg total weekly dose) or placebo (0.9% Sodium Chloride Injection, USP) once a week for 8 weeks starting on Day 1 after all entry criteria had been satisfied. Randomization was based on a computer-generated, site-based randomization schedule, which was distributed to authorized, unblinded personnel at each site. Twenty, 1 mL weekly subcutaneous (SC) injections administered via a ½ inch 30 gauge needle were spaced 4 cm apart on the anterior abdomen between axial planes at +35 mm and −70 mm, relative to the umbilicus. Consistent injection placement was assured by the weekly application of a water-based temporary tattoo defining the location of the 20 administration injection sites. Efficacy endpoints included a Patient- and Clinician-Global Abdominal Perception Scale, a Patient and Clinician Photonumeric Scale (PPnS and CPnS) and duplicate abdominal circumference measurements (taken at +35 mm, 0 and −70 mm, relative to the umbilicus, using a standardized tape measurement (e.g., laser-guided) procedure).

A specific set of standardized instructions (e.g., patient positioning, posture, breathing, etc.) were followed for the collection of all ratings and measurements. Designated clinical site personnel were trained in person to perform each procedure; some procedures also included a training video. Designated clinical site personnel were required to demonstrate that they could perform the procedure prior to study initiation. Specific to the laser-guided tape measure procedure, the reproducibility of the procedure beyond what is obtained with a standard manual tape measure was enhanced with the water-based tattoo on the anterior abdomen, a self-tensioning tape measure, a tripod-mounted laser level and electronic data capture to ensure the independent recording of circumferential measurements. Both the 5-point patient and clinician verbal scales (P-GAPS and C-GAPS) that assigned scores ranging from flat to big (or pronounced) bulge, as well as the 6-point patient and clinician photonumeric scales (PPnS and CPnS) that are a match-to-sample task in which an abdominal profile are selected from a series of 6 identical torsos with a progressively larger anterior abdominal bulge were specifically developed and validated to assess treatment effects in non-obese subjects with central abdominal bulging. Neither patients nor clinicians had access to the others' scale ratings and neither had access to previous scale scores. Final assessments on all outcome measures were performed 1 week after the last injections (Day 57±2 days).

All efficacy analyses used a repeated measures analysis of covariance (ANCOVA) model to determine differences between each active treatment group and placebo. The repeated measures ANCOVA model was performed on change from baseline (CFB) and percent CFB with fixed effects of treatment group, site, P-GAPS at enrollment (“Slight Bulge, Not Flat” or “Bulge” versus “Big Bulge”), time point (class variable), time point by treatment group interaction and baseline value, and a random effect of subject. The compound symmetry structure for the within-subject covariance matrix was used. Differences in the least squares means for each active treatment and placebo were evaluated using Student's t-test at each time point. The assumption of normality for analyses was assessed with the Shapiro-Wilk statistic and residuals were visually examined for heterogeneity via scatter plots. If either assumption was violated, overall treatment differences were assessed using the same repeated measures ANCOVA, but with the response being rank-transformed CFB. For all responder endpoints, the number and percentage of subjects at each response level were summarized by treatment group. Differences between each active treatment group and placebo were evaluated using a van Elteren test at each time point. The influence of CFB weight on treatment outcomes was evaluated in post-hoc analyses. Change from baseline weight was added to and additional ANCOVA model and treatment effects were determined in subsets by CFB weight (≦0 kg, >0 kg).

Demographics & Safety Results: Subjects enrolled in the study were primarily female (81%; 418/513), white (86%; 440/513), with a mean age of 38 (18−71) years and a mean BMI of 23.4 (18.2−29.9) kg/m². Salmeterol xinafoate (0.4 μg (n=132), 1.0 μg (n=124) and 4.0 μg (n=127) total weekly dose) was well tolerated when administered as divided doses (20 SC injections) into abdominal adipose tissue of healthy subjects once a week for 8 consecutive weeks. Ninety-two percent completed the study per protocol; 6 subjects (1%) were lost to follow-up, 4 subjects (<1%) were terminated for protocol violation, 10 subjects (2%) withdrew consent and 21 subjects (4%) withdrew for miscellaneous causes. There were no significant adverse events during this study and no subject discontinued the study due to an adverse event. The most frequently reported treatment-emergent adverse effects related to study drug were mild and transient injection site events, including hematoma, erythema, contusion, and pain. The incidence of these adverse effects is reported in Table 1 (a single incidence (<1%) of an adverse event was reported randomly across organ systems and all treatment groups and is not reported in the table). These injection site events occurred with a similar frequency in subjects in the placebo group and in the salmeterol xinafoate groups and were considered to be related to the typical mechanical trauma of an injection procedure rather than the study drug itself.

TABLE 1
Adverse effects

| | | 0.4 μg | 1.0 μg | 4.0 μg |
|---|---|---|---|---|
| | | Salmeterol | Salmeterol | Salmeterol |
| Adverse Effect | Placebo | Xinafoate | Xinafoate | Xinafoate |
| Any Adverse Event | 10% | 11% | 12% | 12% |
| Definitely or Possibly | | | | |
| Related to Study Drug | | | | |
| Injection Site Hematoma | 2% | 5% | 6% | 6% |
| Injection Site Pain | 2% | 3% | 2% | 2% |
| Injection Site Erythema | 2% | 2% | 2% | 0% |
| Injection Site Hemorrhage | 2% | 0% | 0% | 0% |

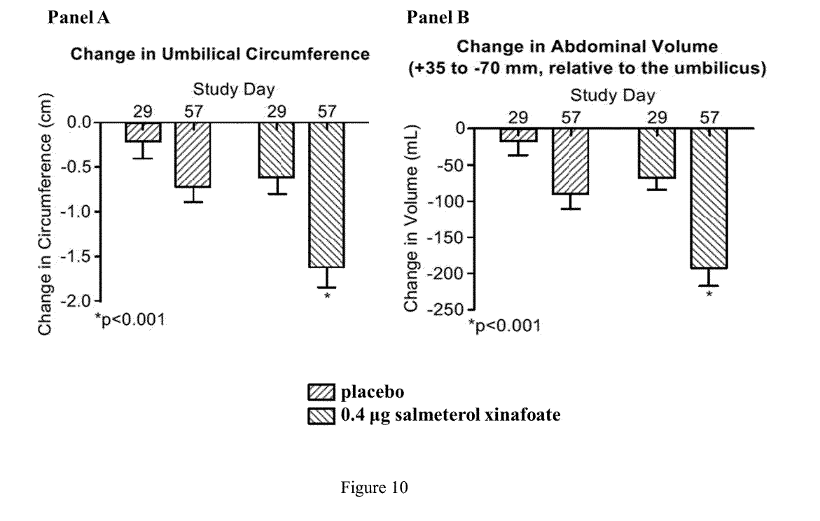

Efficacy Results: Across most outcome measures, the lowest dose of salmeterol xinafoate (0.02 μg/mL; 0.4 μg total weekly dose) was the most efficacious. Significant reductions in mean abdominal circumference assessed by the standardized laser-guided tape measurement procedure were observed with salmeterol xinafoate (0.4 μg) compared to placebo (at the umbilicus, −1.6 cm vs. −0.7 cm, p=0.001; FIG. 10, Panel A) when measured 1 week after the last injections (Day 57). Because circumference was measured at 3 levels on the abdomen, changes in abdominal volume could be calculated using a formula for conical cylinders (volume=(height*pi)/12*(dbottom²+dbottom*dtop+dtop²)). Salmeterol xinafoate (0.4 μg) produced a significant reduction in abdominal volume compared to placebo (−192 mL vs. −90 mL, p=0.001; FIG. 10, panel B).

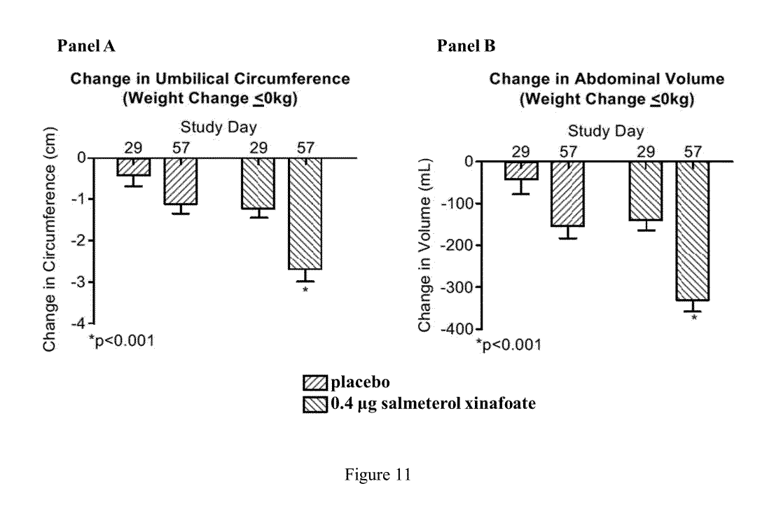

Mean weight change (−0.2 kg) over the course of the trial was similar across all treatment groups. However, weight change itself had an effect on treatment outcomes. Subjects who were weight neutral or lost weight (≦0 kg) from baseline to End-of-Study had enhanced change from baseline and change from placebo treatment effects. For example, in subjects who were weight neutral or lost weight (≦0 kg) in the 0.4 μg total weekly dose group, the mean reduction in umbilical circumference increased from −1.6 cm to −2.7 cm from baseline and from −0.9 cm to −1.6 cm relative to placebo (FIG. 11, panel A). Similarly, in subjects who were weight neutral or lost weight (≦0 kg) in the 0.4 μg total weekly dose group, the mean reduction in abdominal volume (+35 mm to −70 mm, relative to the umbilicus) increased from −192 mL to −329 mL from baseline and from −102 mL to −175 mL relative to placebo (FIG. 11, panel B). No differences in mean weight change (±1.0 kg) were noted between treatment groups when subjects were divided between those who remained weight neutral or lost weight (≦0 kg) and those who gained weight (>0 kg) over the course of the study again showing that this modest weight change cannot account for the treatment effects or differences from placebo. Inclusion or exclusion of weight as a factor within the repeated measures ANCOVA statistical model had no effect on the significance of the treatment effects compared to placebo.

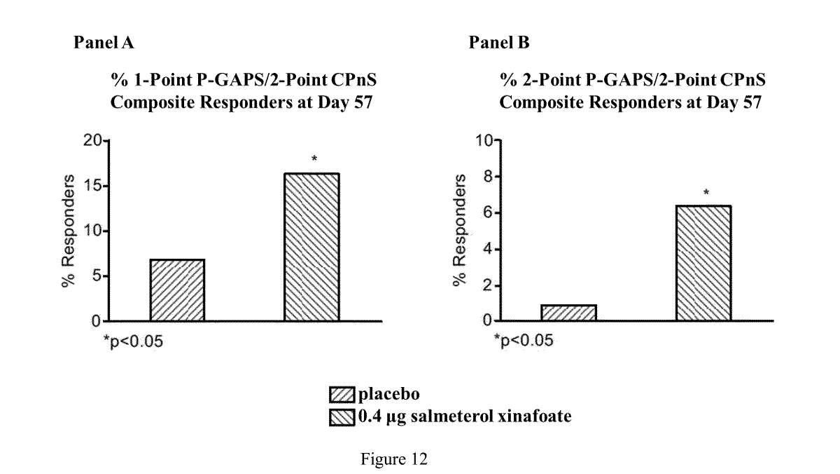

Efficacy was also evaluated using rating scales validated for the purpose of assessing changes in central abdominal bulging. Subjects in the salmeterol xinafoate 0.4 μg total weekly dose treatment group showed consistent mean reductions from baseline across all rating instruments (P-GAPS, C-GAPS, PPnS, CPnS). One way to define treatment benefit is to identify responders on each scale (e.g., a 1- or 2-point (grade) improvement). To further ensure that treatment effects are real, composite analyses are often performed. In the composite, for a subject to be considered a responder, both the patient and the clinician are required to report an improvement. For example, there were significantly more responders on salmeterol xinafoate (0.4 μg) compared to placebo on a composite that requires a subject to report a 2-point (grade) improvement on the P-GAPS and a clinician to report a 2-point improvement on the CPnS for that same subject (FIG. 12, panel B).

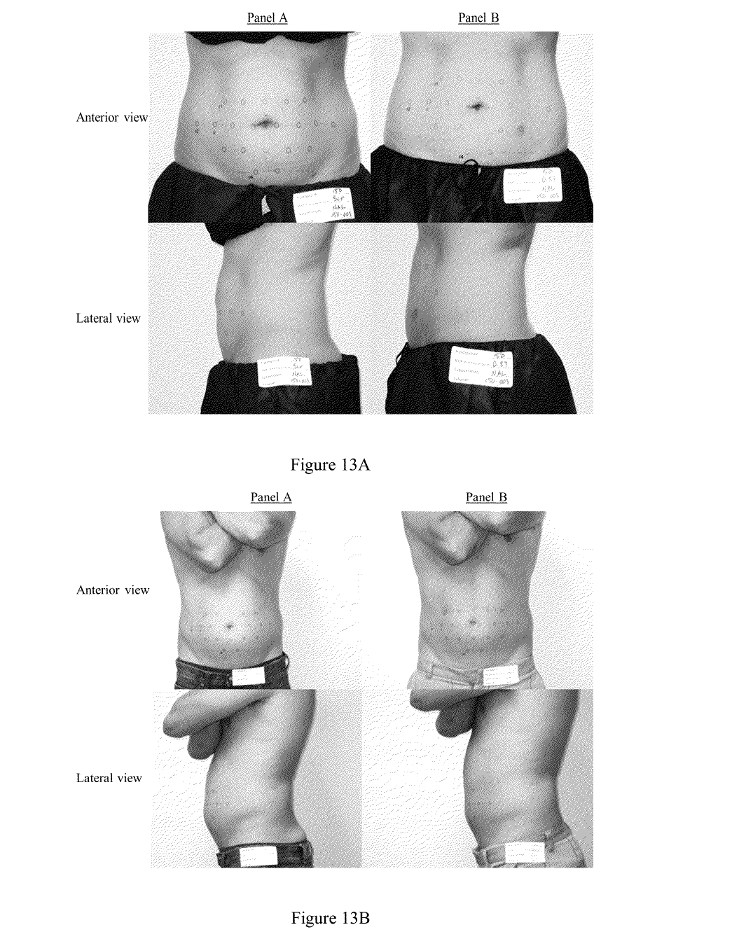

With a composite responder defined as a 1-point improvement on the P-GAPS and a 2-point improvement on the CPnS, the % responders to salmeterol xinafoate (0.4 μg) climbs to 16% (FIG. 12, panel A) and to 22% among subjects who were weight neutral or lost weight (≦0 kg) over the course of the study. Photographs of two salmeterol xinafoate treatment responders are presented in FIGS. 13A (female) and 13B (male) showing anterior and lateral views at screening (panel A) and study endpoint, Day 57 (panel B).

Discussion: Central abdominal bulging is common in non-obese men and women. Although both patients and clinicians use a variety of terms like “pooch” and “rolls” that is “jiggly” and “flabby” to describe the condition, a clinically-relevant diagnosis of central abdominal bulging can be defined using a specific set of diagnostic criteria. In some embodiments, the hallmarks of the central abdominal bulging diagnosis include a focal bulge centered on the umbilicus, often flanked by flat or concave lateral areas, with up to approximately 8 cm of palpable subcutaneous fat in non-obese subjects. Although this condition is common across all demographic groups, the degree to which it is considered to be problematic is highly dependent on individual perception. As a result of central abdominal bulging, some individuals concerns over physical appearance or body image regarding the abdominal bulging leads to impairment in self-image, confidence and overall daily functioning due to social inhibition and anxiety. Treatment options have traditionally been limited to surgical liposuction. However, as with any surgical procedure, there is risk of morbidity and mortality. In addition, there is considerable recovery time, as well as time to aesthetic benefit. Many of the newer energy-based medical devices are non-invasive, but due to the inflammatory process initiated by the ablation of fat cells, recovery time between treatment sessions is also required. A composition comprising salmeterol xinafoate is a non-ablative alternative to liposuction and energy-based medical devices for the treatment of central abdominal bulging. In clinical testing, salmeterol xinafoate administered as twenty 1 mL SC injections once a week for 8 weeks produced significant reductions in abdominal circumference and volume compared to placebo that was recognized by both subjects and clinicians. Significantly, this was achieved without anesthetics/painkillers, “down time” or risk of contour irregularities. Although very small mean weight changes were observed in the clinical study, treatment effects were enhanced in those subjects who remained weight neutral or who lost weight. Treatment effects were observed with only mild and transient injection site reactions that occurred at the same rate as with placebo injections, suggesting that these adverse events are related to the typical mechanical trauma of an injection procedure rather than to the study drug itself.

Conclusions: Central Abdominal Bulging is a common condition in non-obese subjects that can be defined through specific diagnostic criteria. A composition comprising salmeterol xinafoate produced clinically meaningful reductions in central abdominal bulging.

### Example 2

### Patient Self-Assessment

A patient self-assessment is performed by a patient to determine, in whole or in part, suitability for body contouring treatment. The patient self-assessment is a Patient-Global Abdominal Perception Scale (P-GAPS) test. The region of treatment in the patient is the central abdominal region. The P-GAPS is a patient self-assessment of the amount of central abdominal bulging on a 5-point descriptive scale. The patient selects one phrase which best describes the level of central abdominal bulging. The phrases include: flat, almost flat, slight bulge or not flat, bulge and big bulge. A patient describing their central abdominal region as being almost flat, slight bulge or not flat, bulge or big bulge is suitable for body contouring treatment.

The patient self-assessment is optionally performed following a treatment regimen to determine treatment efficacy.

### Example 3

### Patient Abdominal Contour Questionnaire

A patient self-assessment is performed by a patient to determine, in whole or in part, suitability for body contouring treatment. The patient self-assessment is a Patient Abdominal Contour Questionnaire (ACQ). The ACQ comprises the following questions: (1) How important is flattening of the treatment area to you? (2) How self-conscious are you about how the treatment area looks? (3) How much bulging do you see in the treatment area? (4) How bothered are you about the bulging you notice in the treatment area? (5) The treatment area makes me look less attractive? (6) I wear certain clothes to hide or disguise how the treatment area looks? (7) If other people saw the treatment area, I think they would judge me negatively? (8) Because of the bulging in the treatment area, I feel self-conscious when wearing certain types of clothing? (9) The bulge in the treatment area limits the clothes I can buy or wear? (10) Overall, how satisfied are you with the flatness of the treatment area?

A patient completes the ACQ which is then evaluated by clinician to determine, in whole or in part, patient suitability for a body contouring treatment comprising the administration of a beta-2 adrenergic receptor agonist. The patient self-assessment is optionally performed following a treatment regimen to determine treatment efficacy. Example 4: Treatment of submental fat: a randomized, placebo-controlled, clinical trial

Methods: A randomized, double blind, placebo-controlled study is conducted to evaluate the safety and efficacy of a composition comprising the long-acting beta-2 receptor agonist salmeterol xinafoate. The study enrolls adult subjects displeased with the contour of their submental region due to the accumulation of excess subcutaneous fat. Subjects are assessed for suitability by a clinician who ensures that the submental region comprises excess submental fat as measured by magnetic resonance imaging.

Subjects are randomized to receive doses of salmeterol xinafoate (0.1 to 1 μg total weekly dose) or placebo (0.9% sodium chloride injection, USP) once a week for 8 weeks, starting on Day 1, after all entry criteria has been satisfied. One to about fifty weekly subcutaneous injections are administered to the submental region, with each injection spaced at least 0.5 cm apart. Consistent injection placement is assured by the weekly application of a water-based temporary tattoo defining the location of the injection sites.

Results: Subjects enrolled in the study are assessed for treatment efficacy 1 week after the last injections (Day 57±2 days). The assessments include measurement of the submental region by magnetic resonance imaging to evaluate a reduction in submental fat volume compared to placebo.

### Example 5

### Treatment of Exophthalmos: a Randomized, Placebo-Controlled, Clinical Trial

Methods: A randomized, double blind, placebo-controlled study is conducted to evaluate the safety and efficacy of a composition comprising the long-acting beta-2 receptor agonist salmeterol xinafoate and fluticasone propionate. The study enrolls adult subjects having exophthalmos due to thyroid-related eye disease.

Subjects are randomized to receive 1 to 50 doses of salmeterol xinafoate (0.1 to 1 μg total weekly dose) and fluticasone propionate or placebo (0.9% sodium chloride injection, USP) once a week for 8 weeks, starting on Day 1, after all entry criteria has been satisfied. Weekly injections are administered to the orbit.

Results: Subjects enrolled in the study are assessed for treatment efficacy 1 week after the last injections (Day 57±2 days). The assessments include visual assessment of exophthalmos and measurement of proptosis using a Hertel exophthalmometer to determine a reduction in exophthalmos compared to placebo.

### Example 6

### Patient Contour Questionnaire

A patient self-assessment is performed by a patient to determine, in whole or in part, suitability for body contouring treatment. The patient self-assessment is a Patient Contour Questionnaire (CQ). The CQ comprises the following questions: (1) How important is flattening of the treatment area to you? (2) How self-conscious are you about how the treatment area looks? (3) How much bulging do you see in the treatment area? (4) How bothered are you about the bulging you notice in the treatment area? (5) Does the treatment area make you look less attractive? (6) I wear certain clothes to hide or disguise how the treatment area looks? (7) If other people saw the treatment area, I think they would judge me negatively? (8) Because of the contour of the treatment area, I feel self-conscious when wearing certain types of clothing? (9) The contour of the treatment area limits the clothes I can buy or wear? (10) Overall, how satisfied are you with the contour of the treatment area?

A patient completes the CQ which is then evaluated by clinician to determined, in whole or in part, patient suitability for a body contouring treatment comprising the administration of a beta-2 adrenergic receptor agonist. The patient self-assessment is optionally performed following a treatment regimen to determine treatment efficacy.

What is claimed is:

1. A method for determining patient suitability for a body contouring treatment, the method comprising performing one or more assessments, provided that the one or more assessments comprises:
a) determining if the patient is non-obese; and,
b) determining if the patient has subcutaneous fat in a treatment region of the patient's body; provided that the body contouring treatment comprises administration of a composition comprising a long-acting beta-2 adrenergic receptor agonist to the treatment region.

2. The method of claim 1, provided that the one or more assessments further comprises evaluating visible fat protrusion or bulging in the treatment region by selecting a descriptive phrase to describe the visible fat protrusion or bulging from an assessment scale comprising from about 3 to about 10 descriptive phrases that each indicate varying degrees of visible fat protrusion or bulging.

3. The method of claim 2, provided that each descriptive phrase of the assessment scale is converted to a number on an ordinal scale to generate an assessment score; and provided that the lowest assessment score indicates no or a low level of visible fat protrusion or bulging and the highest assessment score indicates a high level of visible fat protrusion or bulging;
and provided that a patient suitable for treatment has an assessment score greater than the lowest assessment score.

4. The method of claim 1, provided that the one or more assessments further comprises performing a match-to-sample task comprising rating visible fat protrusion or bulging in the treatment region of the patient on a photonumeric scale comprising from about 3 to about 10 representative pictures of increasing visible fat protrusion or bulging in corresponding reference treatment regions, each picture having a numeric value directly proportional to amount of visible fat protrusion or bulging; and provided that a patient suitable for treatment has a match-to-task score greater than the lowest numeric value of the photonumeric scale.

5. The method of claim 1, provided that the one or more assessments further comprises measuring the treatment region using a laser-guided manual tape measure; three-dimensional digital photographic imaging of the treatment region; measuring the treatment region using a skin-fold caliper; subjective photographic assessment of the treatment region; administering one or more questionnaires to the patient, a clinician, or both the patient and clinician; performing magnetic resonance imaging of the treatment region; performing ultrasound imaging of the treatment region; visual grading; palpating the treatment region; or a combination thereof.

6. The method of claim 1, provided that the treatment region comprises a submental region, central abdominal region, face, flank, back, chest, arm, leg, buttock, or combination thereof.

7. The method of claim 1, provided that the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof.

8. A method of reducing subcutaneous adipose tissue, the method comprising administering to a treatment region of a patient a composition comprising a long-acting beta-2 adrenergic receptor agonist, provided that the patient has been identified as suitable for reducing subcutaneous adipose tissue or bulging due to subcutaneous adipose tissue in the treatment region as determined by one or more assessments, provided that the one or more assessments comprises:
a) determination that the patient is non-obese; and,
b) determination of subcutaneous fat or bulging in the treatment region.

9. The method of claim 8, provided that the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, salmefamol, or a salt, solvate, polymorph, or combination thereof.

10. The method of claim 8, provided that the composition is administered to the treatment region in one or more sessions, wherein for each session the composition is administered in a total dosage comprising from about 1 nanogram to about 10 micrograms beta-2 adrenergic receptor agonist, and the total dosage is distributed over about 1 to about 100 distinct areas of the region during each session.

11. The method of claim 10, provided that the total dosage of beta-2 adrenergic receptor agonist administered in each session is from about 0.02 micrograms to about 2 micrograms.

12. The method of claim 10, provided that the distance between two adjacent distinct areas of the region is from about 0.5 cm to about 10 cm.

13. The method of claim 8, provided that the treatment region is defined by application of a temporary tattoo or stencil to the treatment region; provided that the temporary tattoo or stencil is configured to facilitate the administration of the composition to distinct areas of the region; and provided that the tattoo or stencil indicates from about 1 to about 100 distinct areas.

14. The method of claim 8, provided that the treatment region comprises a submental region, central abdominal region, back, flank, face, chest, arm, leg, buttock, or combination thereof.

15. A method of determining an effect of a body contouring treatment to a treatment region of a patient's body, the method comprising performing one or more assessments after administration of a composition comprising an effective amount of a long-acting beta-2 adrenergic receptor agonist to the treatment region, provided that the treatment region comprises subcutaneous adipose tissue or bulging due to subcutaneous adipose tissue prior to administration and provided that the patient is non-obese.

16. The method of claim 15, provided that the one or more assessments comprises evaluating visible fat protrusion or bulging in the treatment region by selecting a descriptive phrase to describe the visible fat protrusion or bulging from an assessment scale comprising from about 3 to about 10 descriptive phrases that each indicate varying degrees of visible fat protrusion or bulging.

17. The method of claim 16, provided that each descriptive phrase of the assessment scale is converted to a number on an ordinal scale to generate an assessment score; and provided that the lowest assessment score indicates no or a low level of visible fat protrusion or bulging and the highest assessment score indicates a high level of visible fat protrusion or bulging;
and provided that the evaluation is performed both prior to and after the body contouring treatment and provided that the body contouring treatment is effective if the assessment score after the treatment is lower than the assessment score prior to treatment.

18. The method of claim 15, provided that the one or more assessments comprises performing a match-to-sample task comprising rating visible fat protrusion in the treatment region of the patient on a photonumeric scale comprising from about 3 to about 10 representative pictures of increasing visible fat protrusion or bulging in corresponding reference treatment regions, each picture having a numeric value directly proportional to amount of visible fat protrusion or bulging; and provided that the match-to-sample task is performed both prior to and after the body contouring treatment and provided that the body contouring treatment is effective if the numeric value from the photonumeric scale after treatment is at least 1 or 2 values lower than the numeric value from the photonumeric scale prior to treatment.

19. The method of claim 15, provided that the one or more assessments comprises measuring a visible protrusion of subcutaneous adipose tissue or bulging of the treatment region using a laser-guided manual tape measure; three-dimensional digital photographic imaging of the treatment region; measuring subcutaneous fat of the treatment region using a skin-fold caliper; subjective photographic assessment of the treatment region; administering one or more questionnaires to the patient, a clinician, or both the patient and clinician; performing magnetic resonance imaging of the treatment region; performing ultrasound imaging of the treatment region; visual grading; palpating the treatment region; or a combination thereof;
and provided that after administration of the composition, the protrusion or bulging decreases.

20. The method of claim 15, provided that the beta-2 adrenergic receptor agonist comprises salmeterol, formoterol, bambuterol, eformoterol, indacaterol, clenbuterol, arformoterol, QAB-149, CHF-4226, TA-2005, GSK-159797, GSK-642444, carmoterol, LAS100977, PF-610355, olodaterol, vilanterol, GSK-597901, GSK-159802, GSK-678007, GW642444, or a salt, solvate, polymorph, or combination thereof.
